(19) United States
(12) Patent Application Publication
(10) Pub. No.: US 2026/0331249 A1
(43) Pub. Date: Oct. 22, 2026

(54) MACHINE-LEARNING BASED SYSTEM AND METHOD FOR VALIDATING PRESCRIPTIONS OF PATIENTS

(71) Applicant: Greenfieldworx LLC, Austin, TX (US)

(72) Inventor: Mark Erwin, Austin, TX (US)

(21) Appl. No.: 19/727,256

(22) Filed: Jul. 1, 2026

Related U.S. Application Data
(63) Continuation-in-part of application No. 18/890,824, filed on Sep. 20, 2024.
(60) Provisional application No. 63/637,388, filed on Apr. 23, 2024.

(51) Int. Cl. G16H 20/10; G06F 40/205; G06F 40/30; G16H 10/60

(52) U.S. Cl. CPC G16H 20/10; G06F 40/205; G06F 40/30; G16H 10/60

(57) ABSTRACT

A ML-based system and method for validating prescriptions associated with patients, are disclosed. The ML-based method comprises obtaining the prescriptions including information from databases; storing the prescriptions in electronic medical record (EMR) systems; obtaining data comprising at least one of: information associated with the patients whose prescriptions are filled, and metadata of the filled prescriptions, from pharmacies; matching the filled prescriptions obtained from the pharmacies, with the prescriptions stored in the EMR systems and the matching comprises comparing prescription-reconciliation parameters between filled prescriptions and prescriptions stored in the EMR systems, using machine learning models and cognitive learning models; and validating whether each data of the data associated with the filled prescriptions are compliant with a drug affordability and access initiative program, based on the matching of the filled prescriptions obtained from the pharmacies, with the prescriptions stored in the EMR systems and eligibility parameters prestored in the EMR systems.

### CROSS REFERENCE TO RELATED APPLICATION(S)

The present application is a continuation-in-part of U.S. Patent Application No. 18/890,824, filed Sep. 20, 2024, titled “COMPUTER-IMPLEMENTED SYSTEM AND METHOD FOR VALIDATING PRESCRIPTIONS OF PATIENTS”, which claims the priority of U.S. provisional patent application No. 63/637,388, filed on Apr. 23, 2024, titled “SYSTEM AND METHOD FOR VALIDATING PRESCRIPTION RECONCILIATION PARAMETERS”

### TECHNICAL FIELD

Embodiments of the present disclosure relate to healthcare informatics, and more particularly relate to a machine-learning based (ML-based) system and method for validating one or more prescriptions associated with one or more patients.

### BACKGROUND

Healthcare facilities play a vital role in providing healthcare services to both insured patients and uninsured patients vulnerable to a Human Immunodeficiency Virus (HIV). To support a mission of the healthcare facilities, the healthcare facilities obtain a drug affordability and access initiative certification from a government, thereby allowing the healthcare facilities to earn additional funds for each prescription filled for the insured patients. The additional funds received from pharmacies are crucial for a financial sustainability of the healthcare facilities. However, ensuring a compliance with the drug affordability and access initiative program regulations poses significant challenges. It is essential that the pharmacies accurately report the eligible prescription fills to avoid non-compliance and potential termination of the drug affordability and access initiative program status for the healthcare facilities. Moreover, the healthcare facilities rely heavily on the additional funds, making it imperative to reconcile each prescription filled between pharmacy records and electronic medical records (EMRs) accurately.

Traditional approaches to prescription reconciliation rely on manual data entry and matching, involving cumbersome spreadsheets and time-consuming processes. The healthcare facilities tasked with reconciling prescriptions with the pharmacy records face significant challenges in ensuring an accuracy and the compliance due to the sheer volume of prescriptions processed, the complexity of patient data, and the need to adhere to the stringent drug affordability and access initiative program requirements. The manual reconciliation processes are inherently prone to human error, leading to discrepancies between the pharmacy records and the EMR within healthcare facilities. The errors result in the non-compliance with the drug affordability and access initiative program regulations, potentially leading to financial losses, penalties, and even the termination of the drug affordability.

In the existing technology, a method, and a system of correlating electronic pharmacy data and the EMR are disclosed. The system comprises a correlation module, a first link and a second link to a pharmacy computer network and a second computer network respectively, and a database for storing data from the second computer network. The correlation module, executed by a hardware processor of one or more hardware processors, receives pharmacy queries from the pharmacy computer network and identifies if the pharmacy queries pertain to the EMR in the second computer network. The correlation module converts the pharmacy queries into a protocol of the second computer network and transmits the protocol via the second link. The system also receives electronic medical records (EMR)-based queries from the second computer network, identifies if the EMR-based queries are meant for the pharmacy computer network, converts the EMR-based queries into the protocol of the pharmacy computer network, and transmits the EMR-based queries via the first link. This facilitates seamless communication between the pharmacy data and the EMR. Nevertheless, transmitting the pharmacy queries and the EMR-based queries between the pharmacy computer network and the second computer network via the first link and the second link introduces a communication overhead, potentially leading to delays in query processing and response times.

There are various technical problems with the prescription reconciliation methods in the prior art. In the existing technology, the traditional methods rely on the manual reconciliation processes, requiring a workforce to manually search, match, and reconcile the pharmacy data with the EMR. This is time-consuming and labor-intensive, leading to inefficiencies and increased operational costs. Human involvement in the manual reconciliation processes increases the likelihood of the errors such as data entry mistakes, mismatches, and inconsistencies. The errors lead to inaccuracies in the patient data and financial transactions, compromising patient care and the drug affordability and access initiative program compliance. The manual matching processes are challenging to scale, especially when dealing with large volumes of the pharmacy data and the EMR. As the volume of data increases, a manual workload becomes overwhelming, resulting in bottlenecks and delays in processing. The traditional methods lack robust compliance management mechanisms, making it difficult to ensure adherence to the drug affordability initiative program requirements and regulations.

In addition to the challenges posed by the manual reconciliation process, existing technologies that attempt to automate the prescription reconciliation process fall short of providing comprehensive solutions. Many automated systems lack the sophistication to accurately match prescription data across different datasets, leading to false positives, missed matches, and inefficiencies in the reconciliation workflow. Furthermore, compliance monitoring and reporting within existing systems are limited, leaving the healthcare facilities vulnerable to regulatory scrutiny and audit findings.

Therefore, there is a need for an improved ML-based system and method for validating one or more prescriptions associated with one or more patients, in order to address the aforementioned issues.

### SUMMARY

This summary is provided to introduce a selection of concepts, in a simple manner, which is further described in the detailed description of the disclosure. This summary is neither intended to identify key or essential inventive concepts of the subject matter nor to determine the scope of the disclosure.

In accordance with an embodiment of the present disclosure, a ML-based method for validating one or more prescriptions associated with one or more patients is disclosed. The ML-based method comprises obtaining, by one or more hardware processors, the one or more prescriptions comprising one or more information, from one or more databases. The one or more prescriptions with the one or more information are generated by one or more healthcare providers and stored in the one or more databases. The one or more information in the one or more prescriptions comprise at least one of: name of the one or more patients, date of birth of the one or more patients, one or more dates on which the one or more prescriptions are generated, name of the one or more healthcare providers, name of one or more medication, quantity of the one or more medication, number of refills, and one or more sites at which one or more prescriptions are generated.

The ML-based method further comprises storing, by the one or more hardware processors, the one or more prescriptions in one or more electronic medical record (EMR) systems.

The ML-based method further comprises obtaining, by the one or more hardware processors, one or more data comprising at least one of: information associated with the one or more patients whose prescriptions are filled, and one or more metadata of the one or more filled prescriptions, from one or more pharmacies.

The ML-based method further comprises matching, by the one or more hardware processors, the one or more filled prescriptions obtained from the one or more pharmacies, with the one or more prescriptions stored in the one or more EMR systems. In an embodiment, the matching comprises comparing, by the one or more hardware processors, a plurality of prescription-reconciliation parameters between the one or more filled prescriptions and the one or more prescriptions stored in the one or more EMR systems, using at least one of: one or more machine learning models and one or more cognitive learning models.

In an embodiment, comparing the plurality of prescription-reconciliation parameters between the one or more filled prescriptions and the one or more prescriptions stored in the one or more EMR systems, using the one or more machine learning models, comprises: (a) preprocessing, by the one or more hardware processors, the plurality of prescription-reconciliation parameters by normalizing the plurality of prescription-reconciliation parameters to a standardized format, wherein the plurality of prescription-reconciliation parameters comprise at least one of: a medication description normalized to one or more text fields, and dosage and quantity converted to uniform numerical units; (b) generating, by the one or more hardware processors, a first feature vector representing each of the one or more filled prescriptions and a second feature vector representing each of the one or more prescriptions stored in the one or more EMR systems, using the one or more machine learning models; (c) computing, by the one or more hardware processors, a pairwise similarity score between the first feature vector and the second feature vector, using the one or more machine learning models; (d) aggregating, by the one or more hardware processors, individual similarity scores for each of the plurality of prescription-reconciliation parameters using a plurality of learned weight coefficients to generate a composite match score; (e) classifying, by the one or more hardware processors, each pair of the one or more filled prescriptions and the one or more prescriptions stored in the one or more EMR systems as matched or unmatched based on comparing the composite match score against a learned decision threshold; and (f) updating, by the one or more hardware processors, the plurality of learned weight coefficients based on manually matched mappings to optimize matching accuracy for subsequent comparisons.

In an embodiment, at least one of: the one or more machine learning models and the one or more cognitive learning models, are configured to analyze the plurality of prescription-reconciliation parameters to identify similarities between the one or more filled prescriptions obtained from the one or more pharmacies and the one or more prescriptions stored in the one or more EMR systems.

In an embodiment, for the one or more filled prescriptions obtained from the one or more pharmacies that are not matched with the one or more prescriptions stored in the one or more EMR systems, the matching comprising: (a) learning, by the one or more hardware processors, the manually matched mappings of the one or more filled prescriptions obtained from the one or more pharmacies, with the one or more prescriptions stored in the one or more EMR systems; (b) storing, by the one or more hardware processors, information associated with the manually matched mappings of the one or more filled prescriptions obtained from the one or more pharmacies, with the one or more prescriptions stored in the one or more EMR systems, in a memory; and (c) utilizing, by the one or more hardware processors, the stored information associated with the manually matched mappings of the one or more filled prescriptions obtained from the one or more pharmacies, with the one or more prescriptions stored in the one or more EMR systems.

The ML-based method further comprises validating, by the one or more hardware processors, whether each data of the one or more data associated with the one or more filled prescriptions are compliant with a drug affordability and access initiative program, based on the matching of the one or more filled prescriptions obtained from the one or more pharmacies, with the one or more prescriptions stored in the one or more EMR systems and one or more parameters prestored in the one or more EMR systems.

In an embodiment, comparing the plurality of prescription-reconciliation parameters between the one or more filled prescriptions and the one or more prescriptions stored in the one or more EMR systems, using the one or more cognitive learning models, comprises: (a) parsing, by the one or more hardware processors, the plurality of prescription-reconciliation parameters to extract one or more semantic entities comprising the medication description, the dosage, the quantity, and a unique identifier linked to each medication; (b) constructing, by the one or more hardware processors, a knowledge graph representation linking the extracted semantic entities from the one or more filled prescriptions with corresponding semantic entities from the one or more prescriptions stored in the one or more EMR systems, using the one or more cognitive learning models; (c) performing, by the one or more hardware processors, a semantic reasoning operation on the knowledge graph representation to infer equivalence relationships between the extracted semantic entities, wherein the semantic reasoning operation comprises identifying a generic medication name as equivalent to a corresponding brand-name medication based on a learned ontological relationship; (d) generating, by the one or more hardware processors, a semantic similarity score for each pair of the one or more filled prescriptions and the one or more prescriptions stored in the one or more EMR systems based on the inferred equivalence relationships; and (e) adapting, by the one or more hardware processors, the learned ontological relationships based on the manually matched mappings to optimize semantic reasoning accuracy for subsequent comparisons.

In another embodiment, the ML-based method further comprises training, by the one or more hardware processors, the one or more machine learning models, by: (a) collecting, by the one or more hardware processors, a first training dataset comprising a plurality of labeled prescription pairs, wherein each labeled prescription pair comprises a filled prescription, a corresponding prescription from the one or more EMR systems, and a ground truth label indicating whether each prescription pair is a match or a non-match; (b) extracting, by the one or more hardware processors, a plurality of training feature vectors from the first training dataset, wherein each training feature vector encodes the plurality of prescription-reconciliation parameters; (c) initializing, by the one or more hardware processors, a plurality of weight coefficients corresponding to each of the plurality of prescription-reconciliation parameters; (d) computing, by the one or more hardware processors, a predicted similarity score for each labeled prescription pair by aggregating individual similarity scores using the plurality of weight coefficients; (e) computing, by the one or more hardware processors, a loss value representing a difference between the predicted similarity score and the ground truth label using a loss function; (f) computing, by the one or more hardware processors, a gradient of the loss value with respect to each of the plurality of weight coefficients using backpropagation; and (g) updating, by the one or more hardware processors, each of the plurality of weight coefficients by subtracting a product of a learning rate and the computed gradient from a current value of the weight coefficient.

In yet another embodiment, the ML-based method further comprises training, by the one or more hardware processors, the one or more cognitive learning models, by: (a) ingesting, by the one or more hardware processors, a knowledge corpus comprising a plurality of medication ontologies and generic-to-brand name equivalence tables to establish foundational semantic relationships between medication entities; (b) constructing, by the one or more hardware processors, an initial ontology comprising a plurality of semantic nodes representing medication entities and a plurality of semantic edges representing relationships between the medication entities; (c) parsing, by the one or more hardware processors, a second training dataset comprising the plurality of labeled prescription pairs to extract semantic entities comprising the medication description, the dosage, the quantity, and the unique identifier linked to each medication; (d) populating, by the one or more hardware processors, a knowledge graph with the extracted semantic entities and inferred relationships; (e) learning, by the one or more hardware processors, a plurality of inference rules from the second training dataset, wherein each inference rule defines a logical condition under which two medication entities are determined to be equivalent; (f) validating, by the one or more hardware processors, the learned inference rules by performing semantic reasoning on the knowledge graph and comparing inferred equivalence relationships against ground truth labels; (g) refining, by the one or more hardware processors, the ontological relationships in the knowledge graph based on misclassified prescription pairs; and (h) adapting, by the one or more hardware processors, the ontological relationships based on the manually matched mappings by adding new semantic edges linking the medication entities that are manually determined to be equivalent.

In yet another embodiment, the ML-based method further comprises adapting, by the one or more hardware processors, the one or more pharmacies to generate one or more periodic settlement reports with the one or more data comprising at least one of: the information associated with the one or more patients whose prescriptions are filled, and the one or more metadata of the one or more filled prescriptions.

In yet another embodiment, the ML-based method further comprises storing, by the one or more hardware processors, one or more information associated with at least one of: the one or more healthcare providers, qualified dates of the one or more healthcare providers, the one or more sites at which one or more prescriptions are generated, one or more qualified pharmacies, dates of the one or more qualified pharmacies, name of one or more qualified medicines, and information associated with the one or more filled prescriptions being compliant with the drug affordability and access initiative program.

In yet another embodiment, the ML-based method further comprises triggering, by the one or more hardware processors, one or more events indicating that the one or more prescriptions are logged when the one or more information associated with the one or more prescriptions are compared.

In yet another embodiment, the ML-based method further comprises (a) generating, by the one or more hardware processors, one or more alerts for one or more issues associated with non-compliance of the one or more prescriptions with the drug affordability and access initiative program; (b) performing, by the one or more hardware processors, a compliance checking process when the one or more prescriptions are non-compliant with the drug affordability and access initiative program, wherein the compliance checking process comprises reviewing, by the one or more hardware processors, one or more discrepancies caused by one or more non-compliance prescriptions to determine an adherence to requirements and regulations associated with the drug affordability and access initiative program; and (c) performing, by the one or more hardware processors, one or more actions comprising at least one of: reversing of one or more transactions and implementation of one or more corrective measures to maintain the compliance of the one or more prescriptions with the drug affordability and access initiative program, upon identifying the one or more issues associated with the compliance of the one or more prescriptions with the drug affordability and access initiative program.

In yet another embodiment, the ML-based method further comprises (a) analyzing, by the one or more hardware processors, one or more financial data to validate the one or more financial data to be compliance with the drug affordability and access initiative program, wherein analyzing the one or more financial data comprises assessing one or more factors comprising at least one of: provider eligibility, program eligibility, and location eligibility; and (b) analyzing, by the one or more hardware processors, the one or more factors to determine whether at least one of: the one or more prescriptions and the one or more financial data are compliant with the drug affordability and access initiative program.

In yet another embodiment, the ML-based method further comprises (a) generating, by the one or more hardware processors, a list of issues associated with mappings of the one or more filled prescriptions obtained from the one or more pharmacies, with the one or more prescriptions stored in the one or more EMR systems, for one or more users; and (b) adapting, by the one or more hardware processors, the one or more users to map the one or more filled prescriptions obtained from the one or more pharmacies, with the one or more prescriptions stored in the one or more EMR systems, when the one or more filled prescriptions obtained from the one or more pharmacies, are not matched with the one or more prescriptions stored in the one or more EMR systems.

In one aspect, a ML-based system for validating one or more prescriptions associated with one or more patients, is disclosed. The ML-based system comprises one or more hardware processors and a memory unit. The memory unit is operatively coupled to the one or more hardware processors. The memory unit comprises a plurality of subsystems in form of programmable instructions executable by the one or more hardware processors. The plurality of subsystems comprises a data obtaining subsystem, a storage subsystem, a prescription matching subsystem, a prescription validating subsystem, and an alerts generating subsystem.

The data obtaining subsystem is configured to obtain the one or more prescriptions comprising one or more information, from one or more databases. The one or more prescriptions with the one or more information are generated by one or more healthcare providers and stored in the one or more databases. The one or more information in the one or more prescriptions comprise at least one of: name of the one or more patients, date of birth of the one or more patients, one or more dates on which the one or more prescriptions are generated, name of the one or more healthcare providers, name of one or more medication, quantity of the one or more medication, number of refills, and one or more sites at which one or more prescriptions are generated.

The storage subsystem configured to store the one or more prescriptions in one or more electronic medical record (EMR) systems.

The data obtaining subsystem configured to obtain one or more data comprising at least one of: information associated with the one or more patients whose prescriptions are filled, and one or more metadata of the one or more filled prescriptions, from one or more pharmacies.

The prescription matching subsystem configured to match the one or more filled prescriptions obtained from the one or more pharmacies, with the one or more prescriptions stored in the one or more EMR systems. For comparing the plurality of prescription-reconciliation parameters between the one or more filled prescriptions and the one or more prescriptions stored in the one or more EMR systems, using the one or more machine learning models, the prescription matching subsystem is further configured to compare a plurality of prescription-reconciliation parameters between the one or more filled prescriptions and the one or more prescriptions stored in the one or more EMR systems, using at least one of: one or more machine learning models and one or more cognitive learning models.

For comparing the plurality of prescription-reconciliation parameters between the one or more filled prescriptions and the one or more prescriptions stored in the one or more EMR systems, using the one or more machine learning models, the prescription matching subsystem is further configured to: (a) preprocess the plurality of prescription-reconciliation parameters by normalizing the plurality of prescription-reconciliation parameters to a standardized format, wherein the plurality of prescription-reconciliation parameters comprise at least one of: a medication description normalized to one or more text fields, and dosage and quantity converted to uniform numerical units; (b) generate a first feature vector representing each of the one or more filled prescriptions and a second feature vector representing each of the one or more prescriptions stored in the one or more EMR systems, using the one or more machine learning models; (c) compute a pairwise similarity score between the first feature vector and the second feature vector, using the one or more machine learning models; (d) aggregate individual similarity scores for each of the plurality of prescription-reconciliation parameters using a plurality of learned weight coefficients to generate a composite match score; (e) classify each pair of the one or more filled prescriptions and the one or more prescriptions stored in the one or more EMR systems as matched or unmatched based on comparing the composite match score against a learned decision threshold; and (f) update the plurality of learned weight coefficients based on manually matched mappings to optimize matching accuracy for subsequent comparisons.

In an embodiment, for the one or more filled prescriptions obtained from the one or more pharmacies that are not matched with the one or more prescriptions stored in the one or more EMR systems, the prescription matching subsystem is further configured to: (a) learn the manually matched mappings of the one or more filled prescriptions obtained from the one or more pharmacies, with the one or more prescriptions stored in the one or more EMR systems; (b) store information associated with the manually matched mappings of the one or more filled prescriptions obtained from the one or more pharmacies, with the one or more prescriptions stored in the one or more EMR systems, in a memory; and (c) utilize the stored information associated with the manually matched mappings of the one or more filled prescriptions obtained from the one or more pharmacies, with the one or more prescriptions stored in the one or more EMR systems.

The prescription validating subsystem configured to validate whether each data of the one or more data associated with the one or more filled prescriptions are compliant with a drug affordability and access initiative program, based on the matching of the one or more filled prescriptions obtained from the one or more pharmacies, with the one or more prescriptions stored in the one or more EMR systems and one or more parameters prestored in the one or more EMR systems.

In another aspect, a non-transitory computer-readable storage medium having instructions stored therein that, when executed by a hardware processor, causes the processor to perform method steps as described above.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF DRAWINGS

The disclosure will be described and explained with additional specificity and detail with the accompanying figures in which:

FIG. 1 illustrates an exemplary block diagram representation of a network architecture of a ML-based system for validating one or more prescriptions associated with one or more patients, in accordance with an embodiment of the present disclosure;

FIG. 2 illustrates a detailed view of the ML-based system for validating the one or more prescriptions associated with the one or more patients, such as those shown in FIG. 1, in accordance with an embodiment of the present disclosure;

FIG. 3 illustrates an exemplary flow diagram of the ML-based system for validating the one or more prescriptions associated with the one or more patients, in accordance with an embodiment of the present disclosure;

FIG. 4 illustrates a process flow of the ML-based system for validating the one or more prescriptions associated with the one or more patients, in accordance with an embodiment of the present disclosure; and

FIG. 5A-5C illustrates a flow chart illustrating a ML-based method for validating the one or more prescriptions associated with the one or more patients, in accordance with an embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION OF THE DISCLOSURE

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure. It will be understood by those skilled in the art that the foregoing general description and the following detailed description are exemplary and explanatory of the disclosure and are not intended to be restrictive thereof.

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

The terms “comprise”, “comprising”, or any other variations thereof, are intended to cover a non-exclusive inclusion, such that one or more devices or sub-systems or elements or structures or components preceded by “comprises… a” does not, without more constraints, preclude the existence of other devices, sub-systems, additional sub-modules. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

A computer system (standalone, client or server computer system) configured by an application may constitute a “module” (or “subsystem”) that is configured and operated to perform certain operations. In one embodiment, the “module” or “subsystem” may be implemented mechanically or electronically, so a module include dedicated circuitry or logic that is permanently configured (within a special-purpose processor) to perform certain operations. In another embodiment, a “module” or “subsystem” may also comprise programmable logic or circuitry (as encompassed within a general-purpose processor or other programmable processor) that is temporarily configured by software to perform certain operations.

Accordingly, the term “module” or “subsystem” should be understood to encompass a tangible entity, be that an entity that is physically constructed permanently configured (hardwired) or temporarily configured (programmed) to operate in a certain manner and/or to perform certain operations described herein.

As used herein the term “drug affordability and access initiative program” is a program that assists the certain healthcare organizations, like hospitals and the healthcare facilities that serve low-income and uninsured patients to buy outpatient drugs at discounted prices. The drug affordability and access initiative program aims to reach more patients and provide the patients with affordable medications. The participating healthcare organizations save money on the medications, allowing the healthcare organizations to better serve their communities in need. Herein in exemplary embodiment, the drug affordability and access initiative program comprises a 340B program.

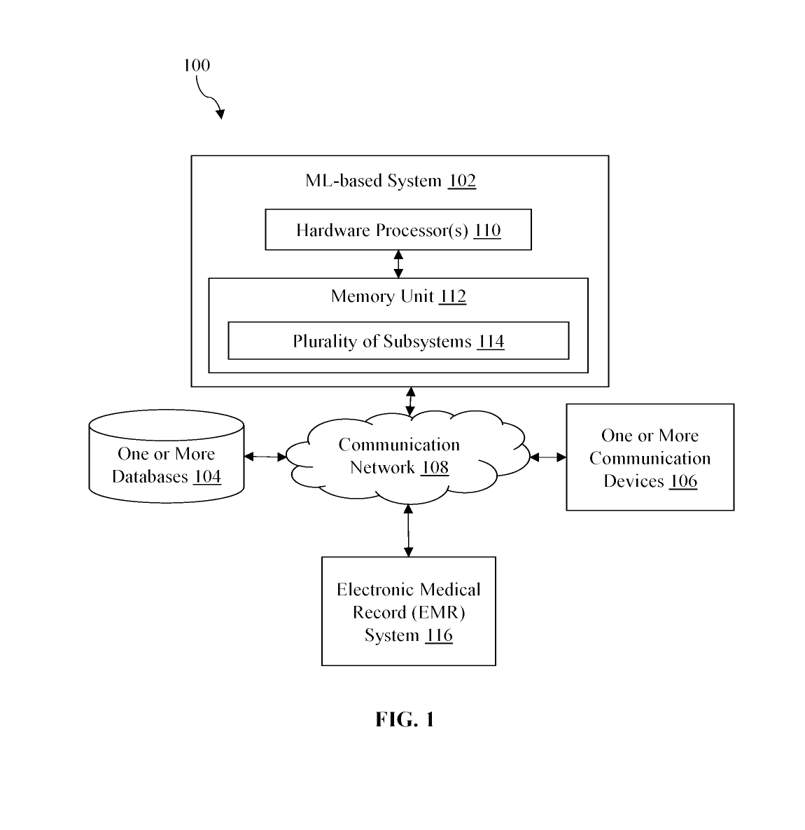

Referring now to the drawings, and more particularly to FIG. 1 through FIG. 5C, where similar reference characters denote corresponding features consistently throughout the figures, there are shown preferred embodiments, and these embodiments are described in the context of the following exemplary system and/or method.

FIG. 1 illustrates an exemplary block diagram representation of a network architecture 100 of a ML-based system 102 for validating one or more prescriptions associated with one or more patients, in accordance with an embodiment of the present disclosure.

According to an exemplary embodiment of the present disclosure, FIG. 1 depicts the network architecture 100 that may include the ML-based system 102, one or more databases 104, and one or more communication devices 106. The ML-based system 102 may be communicatively coupled to the one or more databases 104, and the one or more communication devices 106 via a communication network 108. The communication network 108 may be a wired communication network and/or a wireless communication network. The one or more databases 104 may include, but not limited to, storing, managing, and organizing data related to various aspects of an operation of the ML-based system 102. The data may include comprise, but not limited to, patient data, medication data, pharmacy data, and other relevant data necessary for a functionality of the ML-based system 102. The one or more databases 104 may be any kind of databases including, but not limited to, relational databases, non-relational databases, graph databases, document databases, dedicated databases, dynamic databases, monetized databases, scalable databases, cloud databases, distributed databases, any other databases, and a combination thereof. The one or more databases 104 is configured to support the functionality of the ML-based system 102 and enables efficient data retrieval and storage for various aspects associated with the one or more prescription reconciliation parameters.

The integrated network architecture 100 facilitates seamless communication and data exchange, enabling the ML-based system 102 to operate cohesively for validating the one or more prescriptions associated with the one or more patients. The capability of the ML-based system 102 to validate the one or more prescriptions is underpinned by effective collaboration among the ML-based system 102, the one or more databases 104, and the one or more communication devices 106 within the communication network 108.

The ML-based system 102 is initially configured to obtain the one or more prescriptions including one or more information, from the one or more databases 104. In an embodiment, the one or more prescriptions with the one or more information are generated and provided by one or more healthcare providers and stored in the one or more databases 104. In an embodiment, the one or more information in the one or more prescriptions comprise at least one of: name of the one or more patients, date of birth of the one or more patients, one or more dates on which the one or more prescriptions are generated, name of the one or more healthcare providers, name of one or more medication, quantity of the one or more medication, number of refills, and one or more sites at which one or more prescriptions are generated.

The ML-based system 102 is further configured to store the one or more prescriptions in one or more electronic medical record (EMR) systems 116.

The ML-based system 102 is further configured to obtain one or more data including at least one of: information associated with the one or more patients whose prescriptions are filled, and one or more metadata of the one or more filled prescriptions, from one or more pharmacies.

The ML-based system 102 is further configured to match the one or more filled prescriptions obtained from the one or more pharmacies, with the one or more prescriptions stored in the one or more EMR systems 116. For matching the one or more filled prescriptions obtained from the one or more pharmacies, with the one or more prescriptions stored in the one or more EMR systems 116, the ML-based system 102 is further configured to comparing, by the one or more hardware processors, a plurality of prescription-reconciliation parameters between the one or more filled prescriptions and the one or more prescriptions stored in the one or more EMR systems 116, using at least one of: one or more machine learning models and one or more cognitive learning models.

For comparing the plurality of prescription-reconciliation parameters between the one or more filled prescriptions and the one or more prescriptions stored in the one or more EMR systems 116, using the one or more machine learning models, the ML-based system 102 is configured to: (a) preprocess the plurality of prescription-reconciliation parameters by normalizing the plurality of prescription-reconciliation parameters to a standardized format, wherein the plurality of prescription-reconciliation parameters comprise at least one of: a medication description normalized to one or more text fields, and dosage and quantity converted to uniform numerical units, (b) generate a first feature vector representing each of the one or more filled prescriptions and a second feature vector representing each of the one or more prescriptions stored in the one or more EMR systems 116, using the one or more machine learning models, (c) compute a pairwise similarity score between the first feature vector and the second feature vector, using the one or more machine learning models, (d) aggregate individual similarity scores for each of the plurality of prescription-reconciliation parameters using a plurality of learned weight coefficients to generate a composite match score, (e) classify each pair of the one or more filled prescriptions and the one or more prescriptions stored in the one or more EMR systems 116 as matched or unmatched based on comparing the composite match score against a learned decision threshold, and (f) update the plurality of learned weight coefficients based on manually matched mappings to optimize matching accuracy for subsequent comparisons.

In an embodiment, at least one of: the one or more machine learning models and the one or more cognitive learning models, are configured to analyze the plurality of prescription-reconciliation parameters to identify similarities between the one or more filled prescriptions obtained from the one or more pharmacies and the one or more prescriptions stored in the one or more EMR systems 116.

In an embodiment, for the one or more filled prescriptions obtained from the one or more pharmacies that are not matched with the one or more prescriptions stored in the one or more EMR systems 116, the ML-based system 102 is configured to: (a) learn the manually matched mappings of the one or more filled prescriptions obtained from the one or more pharmacies, with the one or more prescriptions stored in the one or more EMR systems 116, (b) store information associated with the manually matched mappings of the one or more filled prescriptions obtained from the one or more pharmacies, with the one or more prescriptions stored in the one or more EMR systems 116, in a memory, and (c) utilize the stored information associated with the manually matched mappings of the one or more filled prescriptions obtained from the one or more pharmacies, with the one or more prescriptions stored in the one or more EMR systems 116.

The ML-based system 102 is further configured to validate whether each data of the one or more data associated with the one or more filled prescriptions are compliant with a drug affordability and access initiative program, based on the matching of the one or more filled prescriptions obtained from the one or more pharmacies, with the one or more prescriptions stored in the one or more EMR systems 116 and one or more eligibility parameters prestored in the one or more EMR systems 116.

The ML-based system 102 may be hosted on a central server including at least one of: a cloud server or a remote server. In an embodiment, the ML-based system 102 may include at least one of: a user device, a server computer, a server computer over the communication network 108, a cloud-based computing system, a cloud-based computing system over the communication network 108, a distributed computing system, and the like. Further, the communication network 108 may be at least one of: a Wireless-Fidelity (Wi-Fi) connection, a hotspot connection, a Bluetooth connection, a local area network (LAN), a wide area network (WAN), any other wireless network, and the like. In an exemplary embodiment, the one or more communication devices 106 may include, but not limited to, a laptop computer, a mobile device, a Smartphone, a Personal Digital Assistant (PDA), a wearable device, a Smart watch, a tablet computer, a phablet computer, and the like.

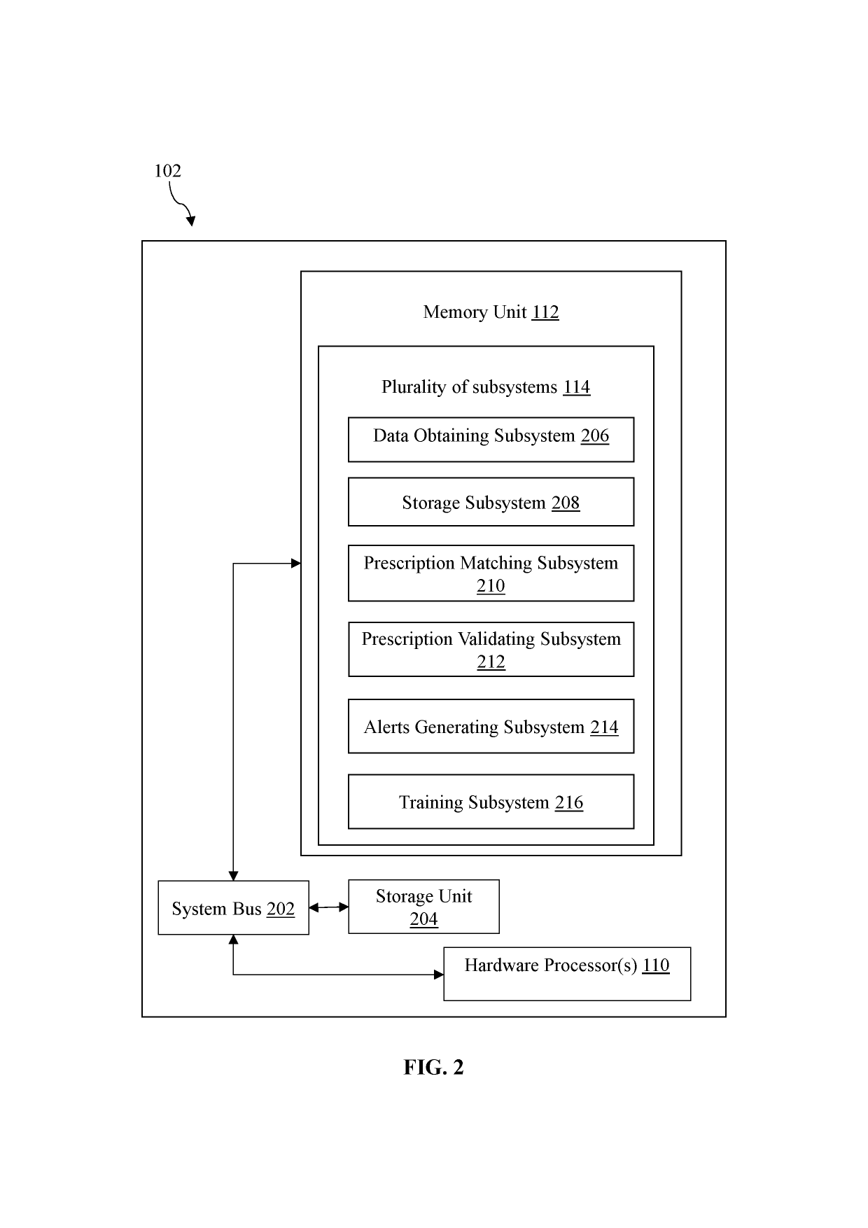

Furthermore, the one or more communication devices 106 may include at least one of: a local browser, a mobile application, and the like. Furthermore, a web application may be used through the local browser and the mobile application to communicate with the ML-based system 102. In an embodiment of the present disclosure, the ML-based system 102 includes a plurality of subsystems 114. Details on the plurality of subsystems 114 have been elaborated in subsequent paragraphs of the present description with reference to FIG. 2.

Though a few components and subsystems are disclosed in FIG. 1, there may be additional components and subsystems which is not shown, such as, but not limited to, ports, routers, repeaters, firewall devices, network devices, databases, network attached storage devices, servers, assets, machinery, instruments, facility equipment, emergency management devices, image capturing devices, any other devices, and combination thereof. A person skilled in the art should not be limiting the components/subsystems shown in FIG. 1.

Those of ordinary skilled in the art will appreciate that the hardware depicted in FIG. 1 may vary for particular implementations. For example, other peripheral devices such as an optical disk drive and the like, local area network (LAN), wide area network (WAN), wireless (e.g., wireless-fidelity (Wi-Fi)) adapter, graphics adapter, disk controller, input/output (I/O) adapter also may be used in addition or place of the hardware depicted. The depicted example is provided for explanation only and is not meant to imply architectural limitations concerning the present disclosure.

Those skilled in the art will recognize that, for simplicity and clarity, the full structure and operation of all data processing systems suitable for use with the present disclosure are not being depicted or described herein. Instead, only so much of the ML-based system 102 as is unique to the present disclosure or necessary for an understanding of the present disclosure is depicted and described. The remainder of the construction and operation of the ML-based system 102 may conform to any of the various current implementations and practices that were known in the art.

FIG. 2 illustrates a detailed view of the ML-based system 102 for validating the one or more prescriptions associated with the one or more patients, such as those shown in FIG. 1, in accordance with an embodiment of the present disclosure.

In an exemplary embodiment, the ML-based system 102 comprises the one or more hardware processors 110, the memory unit 112, and a storage unit 204. The one or more hardware processors 110, the memory unit 112, and the storage unit 204 are communicatively coupled through a system bus 202 or any similar mechanism. The memory unit 112 is operatively coupled to the one or more hardware processors 110. The memory unit 112 comprises the plurality of subsystems 114 in the form of programmable instructions executable by the one or more hardware processors 110.

In an exemplary embodiment, the plurality of subsystems 114 comprises a data obtaining subsystem 206, a storage subsystem 208, a prescription matching subsystem 210, a prescription validating subsystem 212, an alerts generating subsystem 214, and a training subsystem 216.

The one or more hardware processors 110, as used herein, means any type of computational circuit, such as, but not limited to, a microprocessor unit, microcontroller, complex instruction set computing microprocessor unit, reduced instruction set computing microprocessor unit, very long instruction word microprocessor unit, explicitly parallel instruction computing microprocessor unit, graphics processing unit, digital signal processing unit, or any other type of processing circuit. The one or more hardware processors 110 may also include embedded controllers, such as generic or programmable logic devices or arrays, application-specific integrated circuits, single-chip computers, and the like.

The memory unit 112 may be a non-transitory volatile memory and a non-volatile memory. The memory unit 112 may be coupled to communicate with the one or more hardware processors 110, such as being a computer-readable storage medium. The one or more hardware processors 110 may execute machine-readable instructions and/or source code stored in the memory unit 112. A variety of machine-readable instructions may be stored in and accessed from the memory unit 112. The memory unit 112 may include any suitable elements for storing data and machine-readable instructions, such as read-only memory, random access memory, erasable programmable read-only memory, electrically erasable programmable read-only memory, a hard drive, a removable media drive for handling compact disks, digital video disks, diskettes, magnetic tape cartridges, memory cards, and the like. In the present embodiment, the memory unit 112 includes the plurality of subsystems 114 stored in the form of machine-readable instructions on any of the above-mentioned storage media and may be in communication with and executed by the one or more hardware processors 110.

The storage unit 204 may be a cloud storage or the one or more databases 104 such as those shown in FIG. 1. The storage unit 204 may be any kind of the one or more databases 104 such as, but not limited to, relational databases, dedicated databases, dynamic databases, monetized databases, scalable databases, cloud databases, distributed databases, any other databases, and a combination thereof.

The plurality of subsystems 114 includes the data obtaining subsystem 206 that is communicatively connected to the one or more hardware processors 110. The data obtaining subsystem 206 is configured to obtain the one or more prescriptions including one or more information from one or more databases 104. In an embodiment, the one or more prescriptions with the one or more information are generated by one or more healthcare providers and stored in the one or more databases 104. In an embodiment, the one or more information in the one or more prescriptions may include at least one of: name of the one or more patients, date of birth of the one or more patients, one or more dates on which the one or more prescriptions are generated, name of the one or more healthcare providers, name of one or more medication, quantity of the one or more medication, number of refills, and one or more sites at which one or more prescriptions are generated.

The plurality of subsystems 114 includes the storage subsystem 208 that is communicatively connected to the one or more hardware processors 110. The storage subsystem 208 is configured to store the one or more prescriptions in one or more electronic medical record (EMR) systems 116.

The data obtaining subsystem 206 is further configured to obtain the one or more data including at least one of: the information associated with the one or more patients whose prescriptions are filled, and the one or more metadata of the one or more filled prescriptions, from the one or more pharmacies. Prior to obtaining the one or more data from the one or more pharmacies, the one or more pharmacies are configured to fill the one or more prescriptions associated with the one or more patients. The one or more pharmacies are configured to generate one or more periodic settlement reports (e.g., a monthly settlement report) with the one or more data including at least one of: the information associated with the one or more patients whose prescriptions are filled, and the one or more metadata of the one or more filled prescriptions. The one or more pharmacies are configured to share the one or more periodic settlement reports to the ML-based system 102.

The plurality of subsystems 114 includes the prescription matching subsystem 210 that is communicatively connected to the one or more hardware processors 110. The prescription matching subsystem 210 is configured to match the one or more filled prescriptions obtained from the one or more pharmacies, with the one or more prescriptions stored in the one or more EMR systems 116. For matching the one or more filled prescriptions obtained from the one or more pharmacies, with the one or more prescriptions stored in the one or more EMR systems 116, the prescription matching subsystem 210 is configured to compare a plurality of prescription-reconciliation parameters between the one or more filled prescriptions and the one or more prescriptions stored in the one or more EMR systems 116, using at least one of: one or more machine learning models and one or more cognitive learning models.

As used herein, the term "comparing a plurality of prescription-reconciliation parameters using at least one of one or more machine learning models and one or more cognitive learning models" refers to a computational process wherein the machine learning models perform statistical pattern-based matching by generating feature vectors from prescription-reconciliation parameters (such as medication description, dosage, quantity, and unique identifiers), computing pairwise similarity scores between the feature vectors, aggregating the similarity scores using learned weight coefficients to generate a composite match score, and classifying prescription pairs as matched or unmatched based on comparing the composite match score against a learned decision threshold, while the cognitive learning models perform knowledge-based semantic matching by parsing the prescription-reconciliation parameters to extract semantic entities, constructing a knowledge graph representation linking the extracted entities, performing semantic reasoning operations to infer equivalence relationships (such as identifying a generic medication name as equivalent to a corresponding brand-name medication based on learned ontological relationships), and generating semantic similarity scores based on the inferred equivalences. For example, when comparing a filled prescription for "Atorvastatin 20mg" from a pharmacy with a prescription for "Lipitor 20mg" stored in an EMR system 116, the machine learning model may generate feature vectors encoding the medication description, dosage, and quantity, compute a similarity score indicating a partial match due to differing medication names, and apply learned weight coefficients to generate a composite match score, while alternatively, the cognitive learning model may parse both prescriptions to extract semantic entities, construct a knowledge graph linking "Atorvastatin" and "Lipitor," perform semantic reasoning to infer that "Atorvastatin" is the generic equivalent of the brand-name "Lipitor" based on a learned ontological relationship, and generate a high semantic similarity score indicating a match.

In one aspect, for comparing the plurality of prescription-reconciliation parameters between the one or more filled prescriptions and the one or more prescriptions stored in the one or more EMR systems 116, using the one or more machine learning models, the prescription matching subsystem 210 is configured to:
(a) preprocess the plurality of prescription-reconciliation parameters by normalizing the plurality of prescription-reconciliation parameters to a standardized format, wherein the plurality of prescription-reconciliation parameters comprise at least one of: a medication description normalized to one or more text fields, and dosage and quantity converted to uniform numerical units;
(b) generate a first feature vector representing each of the one or more filled prescriptions and a second feature vector representing each of the one or more prescriptions stored in the one or more EMR systems 116, using the one or more machine learning models;
(c) compute a pairwise similarity score between the first feature vector and the second feature vector, using the one or more machine learning models;
(d) aggregate individual similarity scores for each of the plurality of prescription-reconciliation parameters using a plurality of learned weight coefficients to generate a composite match score;
(e) classify each pair of the one or more filled prescriptions and the one or more prescriptions stored in the one or more EMR systems 116 as matched or unmatched based on comparing the composite match score against a learned decision threshold; and
(f) update the plurality of learned weight coefficients based on manually matched mappings to optimize matching accuracy for subsequent comparisons.

The term "preprocessing the plurality of prescription-reconciliation parameters by normalizing the plurality of prescription-reconciliation parameters to a standardized format" refers to a data transformation process wherein the one or more machine learning models convert heterogeneous prescription data into a uniform representation suitable for computational comparison, wherein the medication description is normalized to one or more text fields by applying text preprocessing operations such as lowercasing, removing special characters, tokenizing medication names into constituent terms, and standardizing abbreviations (e.g., converting "mg" and "milligrams" to a common representation), and wherein the dosage and quantity are converted to uniform numerical units by parsing numeric values from text strings and scaling them to a common unit of measurement (e.g., converting "500 mg" and "0.5 g" to a standardized milligram value of 500). For example, when preprocessing a filled prescription containing "LIPITOR 20MG TAB #30" from a pharmacy and a corresponding EMR prescription containing "Atorvastatin Calcium 20 milligrams tablet, quantity thirty," a Siamese neural network may normalize the medication descriptions by tokenizing and lowercasing both entries to produce standardized text fields ["Lipitor", "20", "mg", "tab"] and ["atorvastatin", "calcium", "20", "mg", "tablet"], convert the dosage values to a uniform numerical unit of 20 milligrams, and convert the quantity values to a uniform numerical value of 30, thereby enabling the subsequent feature vector generation and similarity computation steps to operate on consistently formatted data regardless of the original data entry conventions used by the pharmacy or the EMR system 116.

The term "generating a first feature vector representing each of the one or more filled prescriptions and a second feature vector representing each of the one or more prescriptions stored in the one or more EMR systems 116" refers to a numerical encoding process wherein the one or more machine learning models transform the normalized prescription-reconciliation parameters into fixed-length numerical vectors that capture the semantic and structural characteristics of each prescription, wherein each element of the feature vector corresponds to a specific attribute such as the medication description, the dosage, the quantity, and the unique identifier linked to each medication. For example, a gradient boosting classifier such as XGBoost may generate a first feature vector [0.85, 20.0, 30.0, 12345] for a filled prescription where 0.85 represents a TF-IDF weighted encoding of the medication description "Lipitor tablet," 20.0 represents the normalized dosage in milligrams, 30.0 represents the quantity, and 12345 represents the National Drug Code (NDC), and similarly generate a second feature vector [0.78, 20.0, 30.0, 67890] for a corresponding EMR prescription where 0.78 represents the TF-IDF weighted encoding of "atorvastatin calcium tablet," thereby enabling the subsequent pairwise similarity computation to quantify the degree of match between the two prescriptions based on their numerical representations.

The term "computing a pairwise similarity score between the first feature vector and the second feature vector" refers to a mathematical operation wherein the one or more machine learning models calculate a numerical measure indicating the degree of similarity between two prescription records by applying a distance metric or similarity function to the corresponding feature vectors, wherein the similarity score ranges from 0 (no similarity) to 1 (identical match) or is expressed as a distance value where lower values indicate greater similarity. For example, a Siamese neural network may compute a pairwise similarity score by passing the first feature vector and the second feature vector through twin neural network branches with shared weights to generate embedding vectors, and then computing a cosine similarity of 0.92 between the embedding vectors, indicating a high degree of similarity between the filled prescription for "Lipitor 20mg" and the EMR prescription for "Atorvastatin 20mg," thereby enabling the subsequent aggregation and classification steps to determine whether the prescription pair constitutes a match.

The term "aggregating individual similarity scores for each of the plurality of prescription-reconciliation parameters using a plurality of learned weight coefficients to generate a composite match score" refers to a weighted combination process wherein the one or more machine learning models combine separate similarity scores computed for each prescription-reconciliation parameter (such as the medication description, the dosage, the quantity, and the unique identifier) into a single composite score by multiplying each individual similarity score by a corresponding learned weight coefficient and summing the weighted scores, wherein the learned weight coefficients reflect the relative importance of each parameter in determining a correct match. For example, a random forest classifier may assign learned weight coefficients of 0.4 for medication description similarity, 0.25 for dosage similarity, 0.20 for quantity similarity, and 0.15 for NDC similarity, and then compute a composite match score of 0.87 by aggregating individual similarity scores of [0.85, 1.0, 1.0, 0.6] as (0.4 × 0.85) + (0.25 × 1.0) + (0.20 × 1.0) + (0.15 × 0.6) = 0.34 + 0.25 + 0.20 + 0.09 = 0.88, thereby producing a single numerical value that represents the overall likelihood that the filled prescription matches the EMR prescription.

The term "classifying each pair of the one or more filled prescriptions and the one or more prescriptions stored in the one or more EMR systems 116 as matched or unmatched based on comparing the composite match score against a learned decision threshold" refers to a binary classification operation wherein the one or more machine learning models compare the computed composite match score to a threshold value that has been optimized during training to maximize classification accuracy, and assign a "matched" label if the composite match score equals or exceeds the learned decision threshold, or an "unmatched" label if the composite match score falls below the learned decision threshold. For example, a logistic regression classifier trained on historical prescription matching data may determine a learned decision threshold of 0.75 that maximizes the F1-score on a validation dataset, and then classify a prescription pair with a composite match score of 0.88 as "matched" because 0.88 ≥ 0.75, while classifying another prescription pair with a composite match score of 0.62 as "unmatched" because 0.62 < 0.75, thereby enabling the system to automatically identify which filled prescriptions correspond to which EMR prescriptions without manual intervention.

The term "updating the plurality of learned weight coefficients based on manually matched mappings to optimize matching accuracy for subsequent comparisons" refers to an online learning process wherein the one or more machine learning models adjust the weight coefficients assigned to each prescription-reconciliation parameter based on feedback from manually matched mappings provided by human reviewers, wherein the updating comprises computing a gradient of a loss function representing the difference between predicted match scores and ground truth labels from the manually matched mappings, and adjusting each weight coefficient in a direction that minimizes the loss function. For example, when a human reviewer manually matches a filled prescription for "Metformin HCL 500mg" with an EMR prescription for "Metformin Hydrochloride 500 milligrams" that the system initially classified as "unmatched" due to a low medication description similarity score, a support vector machine (SVM) classifier may update the learned weight coefficient for medication description similarity from 0.4 to 0.35 and increase the weight coefficient for dosage similarity from 0.25 to 0.30, thereby improving the matching accuracy for subsequent comparisons involving similar medication name variations.

The plurality of subsystems 110 further includes the training subsystem 216 that is communicatively connected to the one or more hardware processors 110. The training subsystem 216 is configured to train the one or more machine learning models, by:
(a) collecting a first training dataset comprising a plurality of labeled prescription pairs, wherein each labeled prescription pair comprises a filled prescription, a corresponding prescription from the one or more EMR systems 116, and a ground truth label indicating whether each prescription pair is a match or a non-match;
(b) extracting a plurality of training feature vectors from the first training dataset, wherein each training feature vector encodes the plurality of prescription-reconciliation parameters;
(c) initializing a plurality of weight coefficients corresponding to each of the plurality of prescription-reconciliation parameters;
(d) computing a predicted similarity score for each labeled prescription pair by aggregating individual similarity scores using the plurality of weight coefficients;
(e) computing a loss value representing a difference between the predicted similarity score and the ground truth label using a loss function;
(f) computing a gradient of the loss value with respect to each of the plurality of weight coefficients using backpropagation; and
(g) updating each of the plurality of weight coefficients by subtracting a product of a learning rate and the computed gradient from a current value of the weight coefficient.

The term "collecting a first training dataset comprising a plurality of labeled prescription pairs, wherein each labeled prescription pair comprises a filled prescription, a corresponding prescription from the one or more EMR systems 116, and a ground truth label indicating whether each prescription pair is a match or a non-match" refers to a data acquisition process wherein the one or more hardware processors gather historical prescription records that have been previously verified by human reviewers or domain experts, wherein each labeled prescription pair includes a filled prescription record from a pharmacy (containing medication description, dosage, quantity, and unique identifier), a corresponding prescription record from an EMR system 116, and a binary ground truth label (1 for match, 0 for non-match) indicating whether the two prescriptions refer to the same medication order. For example, when training a gradient boosting classifier such as XGBoost for prescription matching, the training subsystem 216 may collect a first training dataset containing 50,000 labeled prescription pairs, wherein a first labeled prescription pair comprises a filled prescription {medication description: "Lisinopril 10mg tablets", dosage: "10mg", quantity: 30, NDC: "68180-0513-01"} from CVS Pharmacy, a corresponding EMR prescription {medication description: "Lisinopril 10 milligrams oral tablet", dosage: "10 milligrams", quantity: 30, NDC: "68180-0513-01"} from Community Health Center, and a ground truth label of 1 indicating a match, while a second labeled prescription pair comprises a filled prescription for "Metformin 500mg" and an EMR prescription for "Metoprolol 50mg" with a ground truth label of 0 indicating a non-match.

The term "extracting a plurality of training feature vectors from the first training dataset, wherein each training feature vector encodes the plurality of prescription-reconciliation parameters" refers to a feature engineering process wherein the one or more hardware processors transform each labeled prescription pair into a numerical vector representation that captures the similarity characteristics between the filled prescription and the EMR prescription across multiple prescription-reconciliation parameters, wherein each training feature vector encodes computed similarity metrics for the medication description, the dosage, the quantity, and the unique identifier linked to each medication. For example, when training a random forest classifier for prescription matching, the training subsystem 216 may extract a training feature vector [0.85, 1.0, 1.0, 1.0, 0.92] for a labeled prescription pair, wherein 0.85 represents a Jaccard similarity score between the tokenized medication descriptions "lisinopril 10mg tablets" and "lisinopril 10 milligrams oral tablet," 1.0 represents an exact match score for the normalized dosage values (both converted to 10 milligrams), 1.0 represents an exact match score for the quantity values (both equal to 30), 1.0 represents an exact match score for the NDC values (both equal to "68180-0513-01"), and 0.92 represents a character-level edit distance similarity score between the full prescription strings, thereby encoding the prescription-reconciliation parameters as a fixed-length numerical vector suitable for model training.

The term "initializing a plurality of weight coefficients corresponding to each of the plurality of prescription-reconciliation parameters" refers to a model initialization process wherein the one or more hardware processors assign initial numerical values to the weight coefficients that will be applied to each prescription-reconciliation parameter during similarity score aggregation, wherein the weight coefficients determine the relative importance of each parameter (medication description, dosage, quantity, and unique identifier) in computing the composite match score, and wherein the initial values may be set to equal weights, random values, or domain-informed prior values. For example, when training a logistic regression classifier for prescription matching, the training subsystem 216 may initialize a plurality of weight coefficients as w = [0.25, 0.25, 0.25, 0.25] corresponding to medication description similarity, dosage similarity, quantity similarity, and NDC similarity respectively, indicating that each prescription-reconciliation parameter is initially assigned equal importance, or alternatively may initialize the weight coefficients as w = [0.40, 0.20, 0.15, 0.25] based on domain knowledge suggesting that medication description and NDC are more important matching criteria than dosage and quantity, wherein these initial weight coefficients will be iteratively updated during training to optimize matching accuracy.

The term "computing a predicted similarity score for each labeled prescription pair by aggregating individual similarity scores using the plurality of weight coefficients" refers to a forward pass computation wherein the one or more hardware processors calculate a composite match score for each labeled prescription pair by multiplying each individual similarity score (for medication description, dosage, quantity, and unique identifier) by its corresponding weight coefficient and summing the weighted values, wherein the predicted similarity score represents the model's estimate of the likelihood that the prescription pair is a match. For example, when training a neural network classifier for prescription matching, the training subsystem 216 may compute a predicted similarity score for a labeled prescription pair by aggregating individual similarity scores [0.85, 1.0, 1.0, 0.70] using weight coefficients [0.35, 0.25, 0.20, 0.20] as follows: predicted_score = (0.35 × 0.85) + (0.25 × 1.0) + (0.20 × 1.0) + (0.20 × 0.70) = 0.2975 + 0.25 + 0.20 + 0.14 = 0.8875, wherein the predicted similarity score of 0.8875 indicates a high likelihood that the filled prescription matches the EMR prescription, and this predicted score will be compared against the ground truth label to compute the loss value.

The term "computing a loss value representing a difference between the predicted similarity score and the ground truth label using a loss function" refers to an error quantification process wherein the one or more hardware processors calculate a numerical measure of the discrepancy between the model's predicted similarity score and the actual ground truth label for each labeled prescription pair, wherein the loss function may comprise binary cross-entropy loss for classification tasks, mean squared error loss for regression tasks, or contrastive loss for similarity learning tasks, and wherein the loss value indicates how far the model's prediction deviates from the correct answer. For example, when training a Siamese neural network for prescription matching, the training subsystem 216 may compute a loss value using binary cross-entropy loss for a labeled prescription pair with a predicted similarity score of 0.8875 and a ground truth label of 1 (match) as follows: loss = -[1 × log(0.8875) + (1-1) × log(1-0.8875)] = -log(0.8875) = 0.1192, wherein the low loss value of 0.1192 indicates that the model's prediction is close to the ground truth, while for another prescription pair with a predicted similarity score of 0.75 and a ground truth label of 0 (non-match), the loss would be computed as loss = -[0 × log(0.75) + 1 × log(0.25)] = -log(0.25) = 1.386, indicating a higher error that requires weight coefficient adjustment.

The term "computing a gradient of the loss value with respect to each of the plurality of weight coefficients using backpropagation" refers to a gradient computation process wherein the one or more hardware processors calculate the partial derivative of the loss value with respect to each weight coefficient, wherein the gradient indicates the direction and magnitude by which each weight coefficient should be adjusted to reduce the loss value, and wherein backpropagation applies the chain rule of calculus to efficiently compute gradients through the computational graph of the model. For example, when training a multi-layer perceptron (MLP) classifier for prescription matching, the training subsystem 216 may compute gradients of the loss value with respect to each weight coefficient using backpropagation, wherein for a loss value of 1.386 and weight coefficients w = [0.35, 0.25, 0.20, 0.20], the computed gradients may be ∂L/∂w = [-0.85, -1.0, -1.0, -0.70] (scaled by the individual similarity scores), indicating that increasing the weight coefficient for medication description similarity (gradient of -0.85) would decrease the loss more significantly than increasing the weight coefficient for NDC similarity (gradient of -0.70), thereby guiding the model to prioritize adjustments to the most impactful weight coefficients.

The term "updating each of the plurality of weight coefficients by subtracting a product of a learning rate and the computed gradient from a current value of the weight coefficient" refers to a gradient descent optimization process wherein the one or more hardware processors adjust each weight coefficient in the direction that minimizes the loss value, wherein the learning rate is a hyperparameter that controls the step size of each update (typically a small value such as 0.01 or 0.001), and wherein the update rule is expressed as w_new = w_current - (learning_rate × gradient). For example, when training a support vector machine (SVM) classifier for prescription matching, the training subsystem 216 may update the weight coefficients using a learning rate of 0.01 and computed gradients ∂L/∂w = [-0.85, -1.0, -1.0, -0.70] as follows: w_new[0] = 0.35 - (0.01 × -0.85) = 0.35 + 0.0085 = 0.3585, w_new[1] = 0.25 - (0.01 × -1.0) = 0.25 + 0.01 = 0.26, w_new[2] = 0.20 - (0.01 × -1.0) = 0.20 + 0.01 = 0.21, and w_new[3] = 0.20 - (0.01 × -0.70) = 0.20 + 0.007 = 0.207, wherein the updated weight coefficients [0.3585, 0.26, 0.21, 0.207] will produce more accurate predicted similarity scores in subsequent training iterations, and this update process is repeated iteratively across all labeled prescription pairs until the model converges to optimal weight coefficients that minimize the overall loss.

In another aspect, for comparing the plurality of prescription-reconciliation parameters between the one or more filled prescriptions and the one or more prescriptions stored in the one or more EMR systems 116, using the one or more cognitive learning models, the prescription matching subsystem 210 is configured to: (a) parse the plurality of prescription-reconciliation parameters to extract one or more semantic entities comprising the medication description, the dosage, the quantity, and a unique identifier linked to each medication; (b) construct a knowledge graph representation linking the extracted semantic entities from the one or more filled prescriptions with corresponding semantic entities from the one or more prescriptions stored in the one or more EMR systems 116, using the one or more cognitive learning models; (c) perform a semantic reasoning operation on the knowledge graph representation to infer equivalence relationships between the extracted semantic entities, wherein the semantic reasoning operation comprises identifying a generic medication name as equivalent to a corresponding brand-name medication based on a learned ontological relationship; (d) generate a semantic similarity score for each pair of the one or more filled prescriptions and the one or more prescriptions stored in the one or more EMR systems 116 based on the inferred equivalence relationships; and (e) adapt the learned ontological relationships based on the manually matched mappings to optimize semantic reasoning accuracy for subsequent comparisons.

The term "parsing the plurality of prescription-reconciliation parameters to extract one or more semantic entities comprising the medication description, the dosage, the quantity, and a unique identifier linked to each medication" refers to a natural language processing operation wherein the one or more cognitive learning models analyze unstructured or semi-structured prescription text to identify and extract discrete semantic components that represent meaningful medication-related concepts, wherein the parsing comprises tokenizing the prescription text into constituent terms, applying named entity recognition to identify medication names, dosage values, quantity values, and unique identifiers such as National Drug Codes (NDCs), and classifying each extracted term into a predefined semantic category. For example, when parsing a filled prescription containing "Lisinopril-HCTZ 10-12.5mg tablets, qty 30, NDC 68180-0513-01" from a pharmacy, a cognitive learning model such as IBM Watson or Google Knowledge Graph may tokenize the prescription text into individual terms ["Lisinopril-HCTZ", "10-12.5mg", "tablets", "qty", "30", "NDC", "68180-0513-01"], apply named entity recognition to identify "Lisinopril-HCTZ" as a medication description entity, "10-12.5mg" as a dosage entity, "30" as a quantity entity, and "68180-0513-01" as a unique identifier entity, and output a structured semantic representation {medication_description: "Lisinopril-HCTZ", dosage: "10-12.5mg", quantity: 30, unique_identifier: "68180-0513-01"} that can be used for subsequent knowledge graph construction and semantic reasoning operations.

The term "constructing a knowledge graph representation linking the extracted semantic entities from the one or more filled prescriptions with corresponding semantic entities from the one or more prescriptions stored in the one or more EMR systems 116" refers to a graph-based data structure creation process wherein the one or more cognitive learning models organize the extracted semantic entities as nodes and establish semantic edges representing potential relationships between entities from different prescription sources, wherein the construction comprises creating medication entity nodes for each extracted semantic entity, creating relationship edges based on attribute similarity and ontological knowledge, and linking nodes from filled prescriptions to candidate nodes from EMR prescriptions based on shared attributes or inferred equivalences. For example, when constructing a knowledge graph for a filled prescription containing semantic entities {medication description: "Atorvastatin Calcium 20mg", dosage: "20mg", quantity: 30} and an EMR prescription containing semantic entities {medication description: "Lipitor 20mg", dosage: "20mg", quantity: 30}, a cognitive learning model such as Microsoft Cognitive Services or Amazon Neptune may create a first medication node "Atorvastatin Calcium 20mg" with attribute edges linking to dosage node "20mg" and quantity node "30," create a second medication node "Lipitor 20mg" with corresponding attribute edges, establish a candidate relationship edge between the two medication nodes based on matching dosage and quantity attributes, and annotate the candidate relationship edge with a preliminary confidence score of 0.65 indicating potential equivalence pending semantic reasoning verification.

The term "performing a semantic reasoning operation on the knowledge graph representation to infer equivalence relationships between the extracted semantic entities, wherein the semantic reasoning operation comprises identifying a generic medication name as equivalent to a corresponding brand-name medication based on a learned ontological relationship" refers to a logical inference process wherein the one or more cognitive learning models traverse the knowledge graph and apply learned ontological rules to determine whether semantically distinct medication entities represent the same underlying drug, wherein the semantic reasoning comprises querying the knowledge graph for candidate relationship edges, retrieving relevant ontological relationships from a medication ontology, applying inference rules to determine equivalence based on active ingredient matching, therapeutic class membership, or generic-to-brand name mappings, and updating the knowledge graph with inferred equivalence edges. For example, when performing semantic reasoning on a knowledge graph containing medication nodes "Atorvastatin Calcium 20mg" and "Lipitor 20mg" connected by a candidate relationship edge, a cognitive learning model such as IBM Watson Discovery or Google Enterprise Knowledge Graph may query a medication ontology to retrieve the ontological relationship {Atorvastatin: generic_equivalent_of: Lipitor}, apply an inference rule stating "IF medication_A is generic_equivalent_of medication_B AND dosage_A equals dosage_B THEN medication_A is equivalent_to medication_B," determine that "Atorvastatin Calcium 20mg" is equivalent to "Lipitor 20mg" because Atorvastatin is the generic equivalent of Lipitor and both have matching dosage values of 20mg, and update the knowledge graph by converting the candidate relationship edge to a confirmed equivalence edge with a confidence score of 0.95.

The term "generating a semantic similarity score for each pair of the one or more filled prescriptions and the one or more prescriptions stored in the one or more EMR systems 116 based on the inferred equivalence relationships" refers to a quantitative scoring process wherein the one or more cognitive learning models compute a numerical value representing the degree of semantic match between a filled prescription and an EMR prescription based on the equivalence relationships inferred during semantic reasoning, wherein the generating comprises aggregating confidence scores from inferred equivalence edges, weighting the aggregated scores based on the semantic importance of each matched entity, and normalizing the weighted scores to produce a final semantic similarity score ranging from 0 (no semantic match) to 1 (complete semantic match). For example, when generating a semantic similarity score for a filled prescription {medication description: "Atorvastatin Calcium 20mg", dosage: "20mg", quantity: 30, NDC: "68180-0631-09"} and an EMR prescription {medication description: "Lipitor 20mg", dosage: "20mg", quantity: 30, NDC: "00071-0156-23"}, a cognitive learning model such as Wolfram Alpha Knowledgebase or Stardog Enterprise may retrieve the inferred equivalence edge between "Atorvastatin Calcium 20mg" and "Lipitor 20mg" with confidence score 0.95, retrieve exact match scores of 1.0 for dosage and 1.0 for quantity, retrieve a partial match score of 0.70 for NDC based on manufacturer code differences, apply semantic importance weights of 0.40 for medication description, 0.25 for dosage, 0.20 for quantity, and 0.15 for NDC, and compute a final semantic similarity score of (0.40 × 0.95) + (0.25 × 1.0) + (0.20 × 1.0) + (0.15 × 0.70) = 0.38 + 0.25 + 0.20 + 0.105 = 0.935, indicating a high-confidence semantic match.

The term "adapting the learned ontological relationships based on the manually matched mappings to optimize semantic reasoning accuracy for subsequent comparisons" refers to an online learning process wherein the one or more cognitive learning models update the medication ontology and inference rules based on feedback from manually matched mappings provided by human reviewers, wherein the adapting comprises identifying ontological gaps where semantic reasoning failed to infer correct equivalences, adding new semantic edges to the medication ontology linking medication entities that were manually determined to be equivalent, adjusting confidence weights for existing ontological relationships based on validation outcomes, and propagating the new ontological knowledge to related medication entities to enable generalization. For example, when a human reviewer manually matches a filled prescription for "Metoprolol Succinate ER 50mg" with an EMR prescription for "Toprol-XL 50mg" that the cognitive learning model initially classified as unmatched due to a missing ontological relationship, a cognitive learning model such as Franz AllegroGraph or Cambridge Semantics Anzo may identify the ontological gap by determining that no generic_equivalent_of relationship exists between "Metoprolol Succinate ER" and "Toprol-XL" in the current medication ontology, add a new semantic edge {Metoprolol Succinate ER: generic_equivalent_of: Toprol-XL} to the medication ontology with an initial confidence score of 0.90, propagate the new ontological relationship to related medication entities by adding inferred edges for other dosage strengths such as {Metoprolol Succinate ER 25mg: generic_equivalent_of: Toprol-XL 25mg} and {Metoprolol Succinate ER 100mg: generic_equivalent_of: Toprol-XL 100mg}, and update the inference rule confidence weights to increase the weight assigned to active ingredient matching when brand name matching fails, thereby improving semantic reasoning accuracy for subsequent comparisons involving extended-release beta-blocker medications.

In an embodiment, the training subsystem 216 is further configured to train the one or more cognitive learning models, by:
(a) ingesting a knowledge corpus comprising a plurality of medication ontologies and generic-to-brand name equivalence tables to establish foundational semantic relationships between medication entities;
(b) constructing an initial ontology comprising a plurality of semantic nodes representing medication entities and a plurality of semantic edges representing relationships between the medication entities;
(c) parsing a second training dataset comprising the plurality of labeled prescription pairs to extract semantic entities comprising the medication description, the dosage, the quantity, and the unique identifier linked to each medication;
(d) populating a knowledge graph with the extracted semantic entities and inferred relationships;
(e) learning a plurality of inference rules from the second training dataset, wherein each inference rule defines a logical condition under which two medication entities are determined to be equivalent;
(f) validating the learned inference rules by performing semantic reasoning on the knowledge graph and comparing inferred equivalence relationships against ground truth labels;
(g) refining the ontological relationships in the knowledge graph based on misclassified prescription pairs; and
(h) adapting the ontological relationships based on the manually matched mappings by adding new semantic edges linking the medication entities that are manually determined to be equivalent.

The term "ingesting a knowledge corpus comprising a plurality of medication ontologies and generic-to-brand name equivalence tables to establish foundational semantic relationships between medication entities" refers to a knowledge acquisition process wherein the one or more hardware processors import and process structured domain knowledge sources that define hierarchical classifications, therapeutic categories, and equivalence mappings between medications, wherein the medication ontologies comprise taxonomic structures organizing medications by drug class, mechanism of action, and therapeutic indication, and wherein the generic-to-brand name equivalence tables comprise mappings linking generic drug names to their corresponding brand-name counterparts. For example, when training a cognitive learning model such as IBM Watson Discovery or Google Enterprise Knowledge Graph for prescription matching, the training subsystem 216 may ingest a knowledge corpus comprising the RxNorm medication ontology that defines hierarchical relationships such as {Atorvastatin: subclass_of: HMG-CoA Reductase Inhibitor: subclass_of: Lipid-Lowering Agent}, the National Drug Code (NDC) directory that maps NDC identifiers to medication attributes, and a generic-to-brand equivalence table containing mappings such as {Atorvastatin: brand_equivalent: Lipitor}, {Metformin: brand_equivalent: Glucophage}, and {Lisinopril: brand_equivalent: Prinivil, Zestril}, thereby establishing foundational semantic relationships that enable the cognitive learning model to recognize that "Atorvastatin 20mg" and "Lipitor 20mg" refer to therapeutically equivalent medications.

The term "constructing an initial ontology comprising a plurality of semantic nodes representing medication entities and a plurality of semantic edges representing relationships between the medication entities" refers to a knowledge structure creation process wherein the one or more hardware processors build a graph-based representation of medication domain knowledge by creating nodes for each distinct medication entity and edges for each semantic relationship between entities, wherein the semantic nodes represent medication concepts such as drug names, active ingredients, dosage forms, and therapeutic classes, and wherein the semantic edges represent typed relationships such as equivalence, hierarchy, composition, and contraindication. For example, when training a cognitive learning model such as Franz AllegroGraph or Cambridge Semantics Anzo for prescription matching, the training subsystem 216 may construct an initial ontology by creating semantic nodes for medication entities including "Atorvastatin," "Lipitor," "Calcium Salt," "20mg," "Tablet," and "HMG-CoA Reductase Inhibitor," and creating semantic edges representing relationships such as {Atorvastatin: generic_equivalent_of: Lipitor}, {Atorvastatin: has_salt_form: Calcium Salt}, {Atorvastatin: belongs_to_class: HMG-CoA Reductase Inhibitor}, and {Lipitor 20mg: has_dosage_strength: 20mg}, thereby establishing a structured knowledge representation that supports semantic reasoning operations during prescription comparison.

The term "parsing a second training dataset comprising the plurality of labeled prescription pairs to extract semantic entities comprising the medication description, the dosage, the quantity, and the unique identifier linked to each medication" refers to a natural language processing operation wherein the one or more hardware processors analyze prescription text from labeled training examples to identify and extract discrete semantic components that represent meaningful medication-related concepts, wherein the parsing comprises tokenizing prescription text, applying named entity recognition to identify medication names, dosage values, quantity values, and unique identifiers, and classifying each extracted term into a predefined semantic category. For example, when training a cognitive learning model such as Microsoft Cognitive Services or Amazon Comprehend Medical for prescription matching, the training subsystem 216 may parse a labeled prescription pair comprising a filled prescription "Metformin HCL 500mg tablets, qty 60, NDC 00093-7214-01" and an EMR prescription "Metformin Hydrochloride 500 milligrams oral tablet, quantity 60" with a ground truth label of "match," extracting semantic entities {medication_description: "Metformin HCL", active_ingredient: "Metformin", salt_form: "Hydrochloride"} from the filled prescription and {medication_description: "Metformin Hydrochloride", active_ingredient: "Metformin", salt_form: "Hydrochloride"} from the EMR prescription, along with dosage entities "500mg" and "500 milligrams," quantity entities "60" and "60," and unique identifier entity "00093-7214-01."n.

The term "populating a knowledge graph with the extracted semantic entities and inferred relationships" refers to a graph construction process wherein the one or more hardware processors add the semantic entities extracted from the training dataset to the knowledge graph as new nodes and establish edges representing relationships between the extracted entities and existing ontology nodes, wherein the populating comprises linking extracted medication entities to corresponding nodes in the initial ontology, creating new nodes for previously unseen entities, and inferring relationships based on attribute matching and ontological rules. For example, when training a cognitive learning model such as Neo4j Graph Data Science or Stardog Enterprise for prescription matching, the training subsystem 216 may populate the knowledge graph by adding a node for the extracted entity "Metformin HCL 500mg" from a filled prescription, linking this node to the existing ontology node "Metformin" via an {is_formulation_of} edge, linking to the existing node "Hydrochloride" via a {has_salt_form} edge, linking to a dosage node "500mg" via a {has_dosage_strength} edge, and inferring a {potential_match} edge to the EMR prescription node "Metformin Hydrochloride 500 milligrams" based on matching active ingredient and dosage attributes.

The term "learning a plurality of inference rules from the second training dataset, wherein each inference rule defines a logical condition under which two medication entities are determined to be equivalent" refers to a rule induction process wherein the one or more hardware processors analyze patterns in the labeled prescription pairs to derive logical rules that predict when two medication entities should be classified as equivalent, wherein each inference rule comprises a set of antecedent conditions (premises) and a consequent conclusion (equivalence determination), and wherein the rules are learned by identifying common attribute patterns in prescription pairs labeled as matches. For example, when training a cognitive learning model such as IBM Watson Knowledge Studio or Wolfram Alpha Knowledgebase for prescription matching, the training subsystem 216 may learn inference rules such as "IF medication_A has_active_ingredient X AND medication_B has_active_ingredient X AND dosage_A equals dosage_B THEN medication_A is_equivalent_to medication_B" with confidence 0.92, "IF medication_A is_generic_of medication_B AND dosage_A equals dosage_B AND quantity_A equals quantity_B THEN medication_A is_equivalent_to medication_B" with confidence 0.95, and "IF medication_A has_NDC N AND medication_B has_NDC N THEN medication_A is_equivalent_to medication_B" with confidence 0.99, wherein the confidence scores are derived from the proportion of correctly classified prescription pairs in the training dataset that satisfy each rule's antecedent conditions.

The term "validating the learned inference rules by performing semantic reasoning on the knowledge graph and comparing inferred equivalence relationships against ground truth labels" refers to a rule evaluation process wherein the one or more hardware processors apply the learned inference rules to the knowledge graph to infer equivalence relationships between medication entities, compare the inferred equivalences against the ground truth labels from the training dataset, and compute accuracy metrics indicating how well each inference rule predicts correct matches. For example, when training a cognitive learning model such as Ontotext GraphDB or PoolParty Semantic Suite for prescription matching, the training subsystem 216 may validate a learned inference rule "IF medication_A has_active_ingredient X AND medication_B has_active_ingredient X AND dosage_A equals dosage_B THEN medication_A is_equivalent_to medication_B" by applying the rule to 1,000 prescription pairs in the training dataset, inferring 850 equivalence relationships where the rule's antecedent conditions are satisfied, comparing the inferred equivalences against ground truth labels to find 820 true positives (correctly inferred matches), 30 false positives (incorrectly inferred matches), and 50 false negatives (missed matches), and computing validation metrics including precision of 0.965 (820/850), recall of 0.943 (820/870), and F1-score of 0.954.

The term "refining the ontological relationships in the knowledge graph based on misclassified prescription pairs" refers to a knowledge graph correction process wherein the one or more hardware processors identify prescription pairs that were incorrectly classified during validation, analyze the causes of misclassification, and modify the ontological relationships in the knowledge graph to improve classification accuracy, wherein the refining comprises adding missing semantic edges, removing erroneous edges, adjusting relationship confidence weights, and updating inference rule conditions. For example, when training a cognitive learning model such as Diffbot Knowledge Graph or Palantir Foundry Ontology for prescription matching, the training subsystem 216 may identify a misclassified prescription pair where a filled prescription for "Metoprolol Succinate ER 50mg" was incorrectly classified as non-matching with an EMR prescription for "Toprol-XL 50mg" due to a missing generic-to-brand equivalence relationship, refine the knowledge graph by adding a new semantic edge {Metoprolol Succinate ER: generic_equivalent_of: Toprol-XL} with confidence 0.90, update the inference rule "IF medication_A is_generic_of medication_B" to include extended-release formulation matching by adding the condition "OR medication_A is_ER_formulation_of medication_B," and re-validate the updated knowledge graph to confirm that the previously misclassified prescription pair is now correctly classified as a match.

The term "adapting the ontological relationships based on the manually matched mappings by adding new semantic edges linking the medication entities that are manually determined to be equivalent" refers to an online learning process wherein the one or more hardware processors incorporate feedback from human reviewers who manually match prescription pairs that the cognitive learning model failed to automatically match, update the knowledge graph by creating new semantic edges representing the manually established equivalence relationships, and propagate the new relationships to related medication entities to enable generalization. For example, when training a cognitive learning model such as Semantic Web Company PoolParty or TopQuadrant TopBraid for prescription matching, the training subsystem 216 may receive a manually matched mapping where a human reviewer determined that a filled prescription for "Losartan Potassium 50mg" is equivalent to an EMR prescription for "Cozaar 50mg" that the model initially classified as unmatched, adapt the knowledge graph by adding a new semantic edge {Losartan Potassium: generic_equivalent_of: Cozaar} with confidence 0.95, propagate the new relationship by adding inferred edges for other dosage strengths such as {Losartan Potassium 25mg: generic_equivalent_of: Cozaar 25mg} and {Losartan Potassium 100mg: generic_equivalent_of: Cozaar 100mg}, and update the inference rule confidence weights to increase the weight assigned to active ingredient matching for angiotensin receptor blocker (ARB) medications, thereby improving semantic reasoning accuracy for subsequent comparisons involving similar medication classes.

In an embodiment, the prescription matching subsystem 210 is configured to generate a list of issues associated with mappings of the one or more filled prescriptions obtained from the one or more pharmacies, with the one or more prescriptions stored in the one or more EMR systems 116, for one or more users. The term "generate a list of issues associated with mappings of the one or more filled prescriptions obtained from the one or more pharmacies, with the one or more prescriptions stored in the one or more EMR systems 116, for one or more users" refers to an automated issue identification process wherein the prescription matching subsystem 210 detects and catalogs prescription records that could not be automatically matched and compiles a structured list of these unresolved mappings for review by human users such as compliance officers or pharmacy technicians, wherein each issue entry in the list includes information identifying the unmatched filled prescription, the candidate EMR prescriptions that were evaluated, the reason for the matching failure (such as missing patient data, ambiguous medication descriptions, or conflicting dosage values), and a priority level indicating the urgency of resolution. For example, when the prescription matching subsystem 210 processes a monthly settlement report containing 5,000 filled prescriptions from CVS Pharmacy and successfully matches 4,850 prescriptions with corresponding EMR records, the prescription matching subsystem 210 may generate a list of 150 issues for the remaining unmatched prescriptions, wherein a first issue entry identifies filled prescription "RX-2024-78543" for "Metoprolol Succinate ER 50mg, qty 30" for patient "John Smith" with failure reason "Multiple Candidate Matches - 3 EMR prescriptions found with similar medication and patient name but different prescribing dates," a second issue entry identifies filled prescription "RX-2024-78544" for "Lisinopril-HCTZ 10-12.5mg" with failure reason "No Matching EMR Record - Patient not found in EMR system 116," and a third issue entry identifies filled prescription "RX-2024-78545" with failure reason "Ambiguous Medication Description - Generic name does not match any known brand equivalents," thereby providing the one or more users with a comprehensive worklist of prescription mapping issues requiring manual resolution.

The prescription matching subsystem 210 is further configured to adapt the one or more users to map the one or more filled prescriptions obtained from the one or more pharmacies, with the one or more prescriptions stored in the one or more EMR systems 116, manually, when the one or more filled prescriptions obtained from the one or more pharmacies, are not matched with the one or more prescriptions stored in the one or more EMR systems 116. The term "adapt the one or more users to map the one or more filled prescriptions obtained from the one or more pharmacies, with the one or more prescriptions stored in the one or more EMR systems 116, when the one or more filled prescriptions obtained from the one or more pharmacies, are not matched with the one or more prescriptions stored in the one or more EMR systems 116" refers to a user interface facilitation process wherein the prescription matching subsystem 210 provides the one or more users with tools, workflows, and contextual information to enable manual resolution of unmatched prescription mappings, wherein the adapting comprises presenting the unmatched filled prescription alongside candidate EMR prescriptions, displaying relevant patient and medication attributes for comparison, providing selection controls for the user to confirm or reject candidate matches, and capturing the user's mapping decision for storage and subsequent learning by the system. For example, when a compliance officer accesses the list of 150 unmatched prescriptions and selects issue entry "RX-2024-78543" for manual review, the prescription matching subsystem 210 may adapt the user interface to display a split-screen comparison showing the filled prescription details (patient name "John Smith," DOB "03/15/1965," medication "Metoprolol Succinate ER 50mg," quantity "30," fill date "03/10/2024," pharmacy "CVS Store #1234") on the left panel and three candidate EMR prescriptions on the right panel (EMR-001 for "Metoprolol Tartrate 50mg" prescribed on "02/28/2024," EMR-002 for "Metoprolol Succinate ER 50mg" prescribed on "03/05/2024," and EMR-003 for "Metoprolol Succinate ER 25mg" prescribed on "03/01/2024"), highlight the matching and differing attributes using color-coded indicators, provide "Confirm Match," "Reject," and "Flag for Further Review" action buttons for each candidate, and upon the user selecting "Confirm Match" for EMR-002, capture the manual mapping decision for storage in the memory and subsequent utilization by the one or more machine learning models to improve matching accuracy for future prescriptions.

Further, the prescription matching subsystem 210 is configured to learn manually matched mappings of the one or more filled prescriptions obtained from the one or more pharmacies, with the one or more prescriptions stored in the one or more EMR systems 116. The term "learning the manually matched mappings of the one or more filled prescriptions obtained from the one or more pharmacies, with the one or more prescriptions stored in the one or more EMR systems 116" refers to a supervised learning process wherein the one or more hardware processors capture and encode the correspondence relationships established by human reviewers when they manually identify correct matches between unmatched filled prescriptions and EMR prescriptions, wherein the learning comprises extracting discriminative features from the manually matched prescription pairs, identifying patterns that distinguish correct matches from incorrect matches, and updating internal model parameters to recognize similar matching patterns in future comparisons. For example, when a human reviewer manually matches a filled prescription for "Lisinopril 10mg tablets, qty 30" from Pharmacy A with an EMR prescription for "Lisinopril-HCTZ 10-12.5mg tablets, quantity 30" that the system initially classified as unmatched due to differing medication descriptions, a gradient boosting classifier may learn this manually matched mapping by extracting features such as the common active ingredient "Lisinopril," the matching dosage strength "10mg," and the identical quantity "30," computing a feature similarity vector representing the prescription pair, associating the feature similarity vector with a positive match label, and updating its internal decision boundaries to recognize that prescriptions sharing a common active ingredient with matching dosage and quantity should be classified as potential matches even when the full medication descriptions differ.

The prescription matching subsystem 210 is further configured to store information associated with the manually matched mappings of the one or more filled prescriptions obtained from the one or more pharmacies, with the one or more prescriptions stored in the one or more EMR systems 116, in a memory (i.e., the one or more databases 104 of the ML-based system 102). The term "storing information associated with the manually matched mappings of the one or more filled prescriptions obtained from the one or more pharmacies, with the one or more prescriptions stored in the one or more EMR systems 116, in a memory" refers to a data persistence process wherein the one or more hardware processors record and index the manually matched prescription pairs along with their associated metadata, feature representations, and matching context in a structured data store, wherein the stored information comprises unique identifiers linking the filled prescription to the corresponding EMR prescription, the extracted feature vectors representing the prescription pair, the matching criteria that led to the manual match determination, and timestamp information indicating when the manual match was established. For example, when a human reviewer manually matches a filled prescription with prescription ID "RX-2024-78543" for "Metoprolol Succinate ER 50mg" from CVS Pharmacy with an EMR prescription with prescription ID "EMR-2024-12876" for "Metoprolol Tartrate 50mg" from a healthcare facility, a random forest classifier may store this manually matched mapping by persisting a mapping record in a relational database containing the pharmacy prescription ID "RX-2024-78543," the EMR prescription ID "EMR-2024-12876," the extracted feature vector [0.72, 50.0, 30.0, 0.85] representing the medication description similarity, dosage, quantity, and active ingredient similarity respectively, the matching criteria "active ingredient equivalence with dosage match," the pharmacy identifier "CVS-Store-4521," and the timestamp "2024-03-15T14:32:00Z," thereby creating a persistent record that can be retrieved for future matching operations.

The prescription matching subsystem 210 is further configured to utilize the stored information (i.e., historical information associated with the mappings) associated with the manually matched mappings of the one or more filled prescriptions obtained from the one or more pharmacies, with the one or more prescriptions stored in the one or more EMR systems 116, from the memory when the one or more filled prescriptions obtained from the one or more pharmacies, with the one or more prescriptions stored in the one or more EMR systems 116, are not matched. In other words, the prescription matching subsystem 210 is configured to utilize the historical information associated with the mappings to match the mappings automatically rather than putting in a queue to reduce human work/intervention.

The term "utilizing the stored information associated with the manually matched mappings of the one or more filled prescriptions obtained from the one or more pharmacies, with the one or more prescriptions stored in the one or more EMR systems 116" refers to a retrieval and application process wherein the one or more hardware processors query the stored manually matched mappings to automatically resolve future unmatched prescriptions that exhibit similar characteristics, wherein the utilizing comprises retrieving historically matched prescription pairs that share common attributes with a current unmatched prescription, computing similarity scores between the current unmatched prescription and the retrieved historical matches, and applying the established mapping relationships to automatically classify the current prescription pair as matched without requiring additional manual intervention. For example, when a new filled prescription for "Metoprolol Succinate ER 50mg, qty 30" from CVS Pharmacy arrives and initially fails to match with an EMR prescription for "Metoprolol Tartrate 50mg, quantity 30," a support vector machine (SVM) classifier may utilize the stored manually matched mapping by querying the mapping database for historical matches involving "Metoprolol" medications from CVS Pharmacy, retrieving the previously stored mapping record linking "Metoprolol Succinate ER 50mg" to "Metoprolol Tartrate 50mg," computing a similarity score of 0.94 between the current prescription pair and the historical mapping based on matching active ingredient, dosage, quantity, and pharmacy source, determining that the similarity score exceeds a retrieval threshold of 0.85, and automatically classifying the current prescription pair as matched based on the established historical mapping relationship, thereby bypassing the manual review queue and reducing human intervention.

The plurality of subsystems 114 includes the prescription validating subsystem 212 that is communicatively connected to the one or more hardware processors 110. The prescription validating subsystem 212 is configured to validate whether each data of the one or more data associated with the one or more filled prescriptions are compliant with the drug affordability and access initiative program, based on the matching of the one or more filled prescriptions obtained from the one or more pharmacies, with the one or more prescriptions stored in the one or more EMR systems 116 and the one or more eligibility parameters prestored in the one or more EMR systems 116. In an embodiment, the ML-based system 102 is configured to generate a list of compliance related issues which are to be determined by the one or more users (e.g., one or more compliance experts). The non-compliance reimbursements are refunded upon determination of the one or more prescriptions are non-compliant to the drug affordability and access initiative program.

The term "validating whether each data of the one or more data associated with the one or more filled prescriptions are compliant with a drug affordability and access initiative program" refers to a compliance determination process wherein the one or more hardware processors evaluate each filled prescription against program eligibility criteria to determine whether the prescription qualifies for discounted pricing under the drug affordability and access initiative program (such as the 340B program), wherein the validating comprises comparing the matched prescription data against one or more parameters prestored in the one or more EMR systems 116, the prestored parameters comprising eligibility criteria such as provider eligibility indicating whether the prescribing healthcare provider is registered with the program, program eligibility indicating whether the patient qualifies for program benefits based on factors such as insurance status or income level, and location eligibility indicating whether the prescription was generated at a qualified healthcare site. For example, when validating a filled prescription for "Metformin 500mg, qty 60" that has been matched to a corresponding EMR prescription for patient "John Smith" at "Community Health Center," the prescription validating subsystem 212 may retrieve prestored parameters from the EMR system indicating that Community Health Center is a registered 340B covered entity with qualification date "2020-01-15," that the prescribing provider "Dr. Jane Doe" is an authorized prescriber with active status, and that the patient "John Smith" is an eligible outpatient who received care at the covered entity on the prescription date, and then determine that the filled prescription is compliant with the drug affordability and access initiative program because all eligibility criteria are satisfied, thereby enabling the healthcare facility to claim the discounted drug pricing and associated financial benefits for that prescription fill.

The storage subsystem 208 is further configured to store one or more information associated with at least one of: the one or more healthcare providers, qualified dates of the one or more healthcare providers, the one or more sites at which the one or more prescriptions are generated, one or more qualified pharmacies, dates of the one or more qualified pharmacies, name of one or more qualified medicines, and information associated with the one or more filled prescriptions being compliant with the drug affordability and access initiative program.

The prescription validating subsystem 212 is configured to trigger one or more events indicating that the one or more prescriptions are logged when the one or more information associated with the one or more prescriptions are compared.

The prescription validating subsystem 212 is further configured to analyze one or more financial data to validate the one or more financial data to be compliance with the drug affordability and access initiative program. In an embodiment, analyzing the one or more financial data includes assessing one or more factors including at least one of: provider eligibility, program eligibility, and location eligibility. The term "analyze one or more financial data to validate the one or more financial data to be compliance with the drug affordability and access initiative program, wherein analyzing the one or more financial data comprises assessing one or more factors comprising at least one of provider eligibility, program eligibility, and location eligibility" refers to a financial verification process wherein the prescription validating subsystem 212 evaluates monetary transactions and reimbursement claims associated with filled prescriptions against program eligibility criteria to ensure that discounted drug pricing is applied only to qualifying prescriptions, wherein provider eligibility comprises verifying that the prescribing healthcare provider is registered with and authorized to participate in the drug affordability and access initiative program on the date of prescription, wherein program eligibility comprises verifying that the patient qualifies for program benefits based on factors such as patient status (inpatient versus outpatient), insurance coverage, and income level, and wherein location eligibility comprises verifying that the prescription was generated at a qualified healthcare site that is registered as a covered entity under the program. For example, when analyzing financial data for a prescription claim of $150.00 for "Humira 40mg injection" dispensed on Mar. 15, 2024, the prescription validating subsystem 212 may assess provider eligibility by querying the EMR system 116 to verify that the prescribing provider "Dr. Sarah Johnson" with National Provider Identifier (NPI) "1234567890" was an active registered provider at "Community Health Center" on the prescription date, assess program eligibility by verifying that the patient "Robert Williams" was classified as an eligible outpatient who received care at the covered entity within the required 30-day window prior to the prescription fill date, and assess location eligibility by verifying that the prescription was generated at "Community Health Center - Main Campus" which is registered as a 340B covered entity with Health Resources and Services Administration (HRSA) ID "12345678."

The prescription validating subsystem 212 is further configured to analyze the one or more factors to determine whether at least one of: the one or more prescriptions and the one or more financial data are compliant with the drug affordability and access initiative program. The term "analyze the one or more factors to determine whether at least one of the one or more prescriptions and the one or more financial data are compliant with the drug affordability and access initiative program" refers to a compliance determination process wherein the prescription validating subsystem 212 evaluates the assessed eligibility factors in combination to render a final compliance decision for each prescription and its associated financial transaction, wherein the analysis comprises applying program-specific compliance rules to the assessed factors, computing a compliance status based on whether all required eligibility criteria are satisfied, and flagging prescriptions or financial data that fail to meet one or more eligibility requirements for further review or corrective action. For example, when analyzing the assessed factors for a prescription claim for "Truvada 200-300mg tablets" with provider eligibility status "verified," program eligibility status "verified," and location eligibility status "failed" (because the prescription was written at an off-site clinic that is not registered as a 340B covered entity), the prescription validating subsystem 212 may apply the compliance rule "all eligibility factors must be verified for 340B pricing to apply," determine that the prescription is non-compliant with the drug affordability and access initiative program because the location eligibility criterion is not satisfied, update the prescription record status from "pending validation" to "non-compliant - location ineligible," generate a compliance exception record documenting the specific eligibility failure, and trigger an alert to the compliance officer indicating that the financial transaction of $85.00 representing the 340B discount must be reversed and the prescription must be rebilled at the non-340B price.

The plurality of subsystems 114 further includes the alerts generating subsystem 214 that is communicatively connected to the one or more hardware processors 110. The alerts generating subsystem 214 is configured to generate one or more alerts for one or more issues associated with non-compliance of the one or more prescriptions with the drug affordability and access initiative program. The term "generate one or more alerts for one or more issues associated with non-compliance of the one or more prescriptions with the drug affordability and access initiative program" refers to an automated notification process wherein the alerts generating subsystem 214 detects prescription records that fail to satisfy program eligibility requirements and creates corresponding alert notifications to inform relevant stakeholders of the compliance violations, wherein the alerts may be transmitted via electronic communication channels such as email notifications, in-application dashboard alerts, or system-generated reports, and wherein each alert includes information identifying the non-compliant prescription, the specific compliance rule that was violated, and the severity level of the violation. For example, when the alerts generating subsystem 214 detects that a filled prescription for "Metformin 500mg" dispensed by CVS Pharmacy on Mar. 15, 2024 cannot be matched to a valid EMR prescription from a registered 340B covered entity, or when the prescription was filled at a pharmacy that is not a qualified contract pharmacy for the healthcare facility, the alerts generating subsystem 214 may generate an alert notification containing the prescription identifier "RX-2024-78543," the patient name "John Smith," the violation type "Unmatched Prescription - No Valid EMR Record," the severity level "High," and the recommended action "Manual Review Required," and transmit this alert to the compliance officer's dashboard and email inbox for immediate attention.

The alerts generating subsystem 214 is further configured to perform a compliance checking process when the one or more prescriptions are non-compliant with the drug affordability and access initiative program. The compliance checking process includes reviewing one or more discrepancies caused by one or more non-compliance prescriptions to determine an adherence to requirements and regulations associated with the drug affordability and access initiative program. Additionally, a compliance queue serves as a designated area where compliance-related tasks are prioritized and managed systematically, ensuring an effective compliance management within the ML-based system 102.

The term "perform a compliance checking process when the one or more prescriptions are non-compliant with the drug affordability and access initiative program, wherein the compliance checking process comprises reviewing one or more discrepancies caused by one or more non-compliance prescriptions to determine an adherence to requirements and regulations associated with the drug affordability and access initiative program" refers to an automated verification workflow wherein the alerts generating subsystem 214 systematically analyzes each flagged non-compliant prescription to identify the root cause of the compliance failure, wherein the reviewing comprises comparing the prescription data against program eligibility criteria including provider eligibility (whether the prescribing healthcare provider is registered with the program), patient eligibility (whether the patient qualifies for program benefits), location eligibility (whether the prescription was generated at a qualified healthcare site), and pharmacy eligibility (whether the dispensing pharmacy is a qualified contract pharmacy). For example, when the alerts generating subsystem 214 performs a compliance checking process for a flagged prescription for "Lisinopril 10mg" dispensed on Apr. 1, 2024, the alerts generating subsystem 214 may review the discrepancy by retrieving the prescription record from the pharmacy settlement report, querying the EMR system 116 to verify that the prescribing provider "Dr. Jane Doe" was an active registered provider on the prescription date, verifying that the patient "Mary Johnson" was an eligible outpatient who received care at the covered entity within the required timeframe, confirming that the dispensing pharmacy "Walgreens Store #1234" was a qualified contract pharmacy on the fill date, and determining that the compliance failure occurred because the patient's visit to the covered entity occurred 45 days after the prescription fill date, which exceeds the 30-day eligibility window required by program regulations.

In an embodiment, the alerts generating subsystem 214 is configured to perform one or more actions including at least one of: reversing of one or more transactions and implementation of one or more corrective measures to maintain the compliance of the one or more prescriptions with the drug affordability and access initiative program, upon identifying the one or more issues associated with the compliance of the one or more prescriptions with the drug affordability and access initiative program.

The term "perform one or more actions comprising at least one of reversing of one or more transactions and implementation of one or more corrective measures to maintain the compliance of the one or more prescriptions with the drug affordability and access initiative program, upon identifying the one or more issues associated with the compliance of the one or more prescriptions with the drug affordability and access initiative program" refers to an automated remediation process wherein the alerts generating subsystem 214 executes corrective operations to address identified compliance violations, wherein reversing of one or more transactions comprises generating refund requests or credit adjustments to return improperly claimed 340B discounts to the appropriate parties, and wherein implementation of one or more corrective measures comprises updating prescription records, flagging accounts for enhanced monitoring, adjusting eligibility parameters, or initiating workflow processes to prevent future similar violations. For example, when the alerts generating subsystem 214 identifies that a prescription for "Atorvastatin 20mg" was incorrectly claimed under the 340B program because the patient was enrolled in Medicaid (which constitutes a duplicate discount violation), the alerts generating subsystem 214 may perform corrective actions by generating a transaction reversal record that credits the manufacturer $45.00 representing the 340B discount amount that was improperly claimed, updating the prescription record status from "340B Eligible" to "Non-Eligible - Medicaid Duplicate Discount," flagging the patient account "Patient ID 12345" for enhanced eligibility verification on future prescriptions, generating a corrective action report documenting the violation and remediation steps for audit purposes, and implementing a corrective measure that adds an automated eligibility check to verify Medicaid enrollment status before processing future 340B claims for this patient.

In an alternative embodiment, one or more cognitive learning models and one or more machine learning models are incorporated into a comparison process that employs one or more algorithms. The one or more algorithms are configured to identify matches by comparing various data points. The one or more algorithms are configured to analyze the one or more prescription reconciliation parameters. Based on the proprietary one or more algorithms, the ML-based system 102 is configured to efficiently identify similarities between the one or more pharmacy records and the one or more Electronic Medical Record (EMR) systems 116. In another embodiment, the pharmacy records are imported in the ML-based system 102 from the pharmacies in an Excel format.

In other words, at least one of: the one or more machine learning models and the one or more cognitive learning models, are configured to analyze the plurality of prescription-reconciliation parameters to identify similarities between the one or more filled prescriptions obtained from the one or more pharmacies and the one or more prescriptions stored in the one or more EMR systems 116.

The above process is a computational analysis process wherein the machine learning models perform statistical pattern-based similarity identification by extracting feature vectors from prescription-reconciliation parameters (such as medication description, dosage, quantity, and unique identifiers), computing pairwise similarity scores using distance metrics (such as cosine similarity or Euclidean distance), and applying learned weight coefficients to generate composite match scores that quantify the degree of similarity between prescription records, while the cognitive learning models perform knowledge-based semantic similarity identification by parsing prescription-reconciliation parameters to extract semantic entities, constructing knowledge graph representations linking the extracted entities, performing semantic reasoning operations to infer equivalence relationships based on learned ontological relationships (such as generic-to-brand name equivalences), and generating semantic similarity scores based on the inferred equivalences. For example, when analyzing a filled prescription for "Metformin HCL 500mg tablets, quantity 60" from a pharmacy and a prescription for "Metformin Hydrochloride 500 milligrams oral tablet, qty 60" stored in an EMR system 116, a gradient boosting classifier may extract feature vectors encoding the normalized medication description, dosage value of 500 milligrams, and quantity value of 60, compute individual similarity scores of 0.82 for medication description (due to abbreviation differences), 1.0 for dosage, and 1.0 for quantity, apply learned weight coefficients of 0.4, 0.3, and 0.3 respectively to generate a composite match score of 0.928, and classify the prescription pair as "matched" because 0.928 exceeds the learned decision threshold of 0.75, while alternatively, a cognitive learning model may parse both prescriptions to extract semantic entities "Metformin HCL" and "Metformin Hydrochloride," construct a knowledge graph linking these entities, perform semantic reasoning to infer that "HCL" is an abbreviation for "Hydrochloride" based on a learned ontological relationship, and generate a semantic similarity score of 0.98 indicating a high-confidence match.

In an alternative embodiment of the present disclosure, the plurality of subsystems 114 includes the data obtaining subsystem 206 that is communicatively connected to the one or more hardware processors 110. The data obtaining subsystem 206 is configured to serve as a conduit for obtaining one or more patient data, one or more medication data, and one or more pharmacy data from one or more data repository units of the one or more pharmacies and the one or more electronic medical record (EMR) systems 116 associated with the one or more healthcare entities. The one or more patient data, the one or more medication data, and the one or more pharmacy data are obtained through the communication network 108 associated with the one or more communication devices 106. Once obtained, the one or more patient data, the one or more medication data, and the one or more pharmacy data are securely stored within the one or more databases 104 of the ML-based system 102, thereby laying a groundwork for subsequent processing and analysis. This seamless obtaining process ensures that the ML-based system 102 remains well-equipped with up-to-date and comprehensive data essential for effective healthcare management and decision-making.

In an alternative embodiment of the present disclosure, the plurality of subsystems 114 optionally includes a patient data comparing subsystem that is communicatively connected to the one or more hardware processors 110. The patient data comparing subsystem is configured to employ one or more patient data (i.e., one or more first patient data) from the one or more pharmacy records to establish the accurate match of the one or more patient data with one or more patient data (i.e., one or more second patient data) present in the one or more EMR systems 116 associated with the healthcare facilities. The patient data comparing subsystem employs the one or more cognitive learning models, the one or more machine learning models, and data comparison techniques to ensure precise alignment between the one or more patient data (i.e., the one or more first patient data) obtained from the one or more pharmacy records and the one or more patient data (i.e., the one or more second patient data) stored within the one or more EMR systems 116. The patient data comparing subsystem operates by analyzing various patient attributes, including at least one of: names, dates of birth, addresses, and other demographic details, to identify comparison records within the one or more EMR systems 116.

In other words, the patient data comparing subsystem is configured to determine whether the one or more first patient data associated with the one or more pharmacies are compliant with the one or more second patient data associated with the one or more electronic medical record (EMR) systems 116 within the one or more healthcare entities, by comparing the one or more first patient data associated with the one or more pharmacies with the one or more second patient data associated with the one or more electronic medical record (EMR) systems 116, using at least one of: the one or more cognitive learning models, the one or more machine learning models, and the data comparison techniques.

If the patient data comparing subsystem initially fails to find a match, the patient data comparing subsystem flags the one or more patient data for manual review within a matching queue, where a workforce manually reviews the one or more patient data to confirm the correct patient match, ensuring an accuracy and a consistency in the one or more patient data across the ML-based system 102. Once the match is validated, mapping data is created to link the one or more patient data from the one or more pharmacy records to the one or more EMR systems 116 associated with the healthcare facilities. The created mapping data enables the automatic comparison for future records of the same patient from the same pharmacy. Additionally, the established match is retroactively applied to all corresponding records in the matching queue, ensuring the consistency across the one or more patient data and minimizing a manual intervention.

In an alternative embodiment of the present disclosure, the plurality of subsystems 114 optionally includes the medication data comparing subsystem that is communicatively connected to the one or more hardware processors 110. The medication data comparing subsystem is configured to ensure the accurate matching of one or more medication data (i.e., one or more first medication data) between the one or more pharmacy records and one or more medication data (i.e., one or more second medication data) present in the one or more EMR systems 116. In other words, the medication data comparing subsystem is configured to determine whether the one or more first medication data associated with the one or more pharmacies are compliant with the one or more second medication data associated with the one or more electronic medical record (EMR) systems 116 within the one or more healthcare entities, by comparing the one or more first medication data associated with the one or more pharmacies with the one or more second medication data associated with the one or more electronic medical record (EMR) systems 116, using at least one of: the one or more cognitive learning models, the one or more machine learning models, and the data comparison techniques.

Initially, the one or more medication data are compared based on a National Drug Code (NDC), which serves as a unique identifier for each medication. In other words, the one or more first medication data associated with the one or more pharmacies, are compared with the one or more second medication data associated with the one or more electronic medical record (EMR) systems 116, based on information associated with a national drug code (NDC). In an embodiment, the information associated with the national drug code (NDC) is a unique identifier for each medication. However, recognizing that the one or more pharmacies may dispense similar medications that may not precisely match a prescribed drug in the one or more medication data, henceforth the medication data comparing subsystem is configured to incorporate one or more matching criteria (i.e., one or more additional matching criterion).

The one or more matching criteria includes matching medication description, dosage, and quantity to accommodate variations in the medication data dispensed. In other words, the one or more matching criteria including at least one of: the medication description, the dosage, and the quantity, are used to determine similarities between the one or more first medication data associated with the one or more pharmacies and the one or more second medication data associated with the one or more electronic medical record (EMR) systems 116. Furthermore, the medication data comparing subsystem is configured to employ the prior mapping data created during reconciliation efforts or linked NDCs to enhance the matching accuracy. In other words, the medication data comparing subsystem is configured to optimize matching accuracy between the one or more first medication data associated with the one or more pharmacies and the one or more second medication data associated with the one or more electronic medical record (EMR) systems 116, based on at least one of: prior mapped medication data created during a compliance determination process and the national drug code (NDC) linked to each medication.

For instance, a generic medication may be automatically mapped to compare the corresponding brand-name drug, streamlining the comparison process, and reducing the need for the manual intervention. By employing these multiple comparing strategies, the medication data comparing subsystem is configured to ensure the robust and reliable matching of the one or more medication data, thereby enhancing the accuracy and the efficiency of the comparison/matching process.

In an alternative embodiment of the present disclosure, the plurality of subsystems 114 further includes the pharmacy data comparing subsystem that is communicatively connected to the one or more hardware processors 110. The pharmacy data comparing subsystem is configured to ensure the accurate matching of the one or more pharmacy data from the pharmacy records with the one or more pre-stored pharmacy data. In other words, the pharmacy data comparing subsystem is configured to determine whether the one or more pharmacy data are compliant with the one or more pre-stored pharmacy data by comparing the one or more pharmacy data with the one or more pre-stored pharmacy data, using at least one of: the one or more cognitive learning models, the one or more machine learning models, and the data comparison techniques.

The one or more pre-stored pharmacy data are stored within the one or more databases 104 employed for the prescription reconciliation. Additionally, the pharmacy data comparing subsystem is configured to employ other pharmacy information including previously created mapping data to enhance the matching accuracy. Depending on the one or more pharmacy data provided in the pharmacy records, the pharmacy data comparing subsystem determines the most appropriate matching criteria to establish connections between the one or more pharmacy records and the one or more EMR systems 116. By employing these multiple comparing/matching strategies, the pharmacy data comparing subsystem is configured to ensure the robust and reliable matching of the pharmacy data, thereby facilitating the accurate reconciliation and compliance within a drug affordability and access initiative program. In other words, the pharmacy data comparing subsystem is configured to validate the one or more pharmacies in compliant with a drug affordability and access initiative program, based on comparison of the one or more pharmacy data with the one or more pre-stored pharmacy data.

In an alternative embodiment of the present disclosure, the plurality of subsystems 114 further includes the financial information recording subsystem that is communicatively connected to the one or more hardware processors 110. The financial information recording subsystem is configured to compare at least one of: the one or more patient data, the one or more medication data, and the one or more pharmacy data, with one or more prescriptions obtained from the one or more electronic medical record (EMR) systems 116 associated with the one or more healthcare entities. The financial information recording subsystem is further configured to validate accuracy of the one or more prescriptions and one or more financial information based on comparison of at least one of: the one or more patient data, the one or more medication data, and the one or more pharmacy data, with the one or more prescriptions. The financial information recording subsystem is further configured to generate the one or more financial data based on the analysis of the one or more financial information associated with each prescription of the one or more prescriptions.

Moreover, the financial information recording subsystem is configured to maintain the record of expected fills (i.e., one or more records of one or more prescriptions obtained from the one or more electronic medical record (EMR) systems 116), which are reconciled against the actual prescription fills reported by the one or more pharmacies. This proactive approach allows the ML-based system 102 to identify and act on any outstanding prescription fills, facilitating timely reconciliation and ensuring a compliance with the drug affordability and access initiative program requirements. The identification and process of one or more identified outstanding prescriptions may assist in accurately recording how much money is involved with each prescription. Additionally, the ML-based system 102 keeps track of what prescriptions are expected to be filled and compares the prescriptions to actual occurrences for reconciliation. This way, any missing prescriptions are spotted and addressed promptly, ensuring everything stays in line with the rules of the drug affordability and access initiative program.

In an alternative embodiment of the present disclosure, the plurality of subsystems 114 further includes the compliance determining subsystem that is communicatively connected to the one or more hardware processors 110. The compliance determining subsystem is configured to determine whether at least one of: the one or more prescriptions and the one or more financial data are compliant with the drug affordability and access initiative program upon analysis of the one or more financial information associated with each prescription of the one or more prescriptions. The compliance determining subsystem is configured to ensure adherence to the drug affordability and access initiative program regulations once the prescription fill is accurately recorded through the comparison/matching process. In an embodiment, one or more events are triggered in the ML-based system 102 to indicate that the prescription fill is logged when the one or more prescription reconciliation parameters are compared.

In an alternative embodiment of the present disclosure, the plurality of subsystems 114 further includes the report generating subsystem that is communicatively connected to the one or more hardware processors 110. The report generating subsystem is configured to generate one or more reports based on the compliance of at least one of: the one or more prescriptions and the one or more financial data, with the drug affordability and access initiative program. The one or more reports may include, but not limited to, one or more summaries of the one or more prescriptions (i.e., one or more prescription fills), the one or more financial data, one or more compliance statuses of at least one of: the one or more financial data and the one or more prescriptions, and the like.

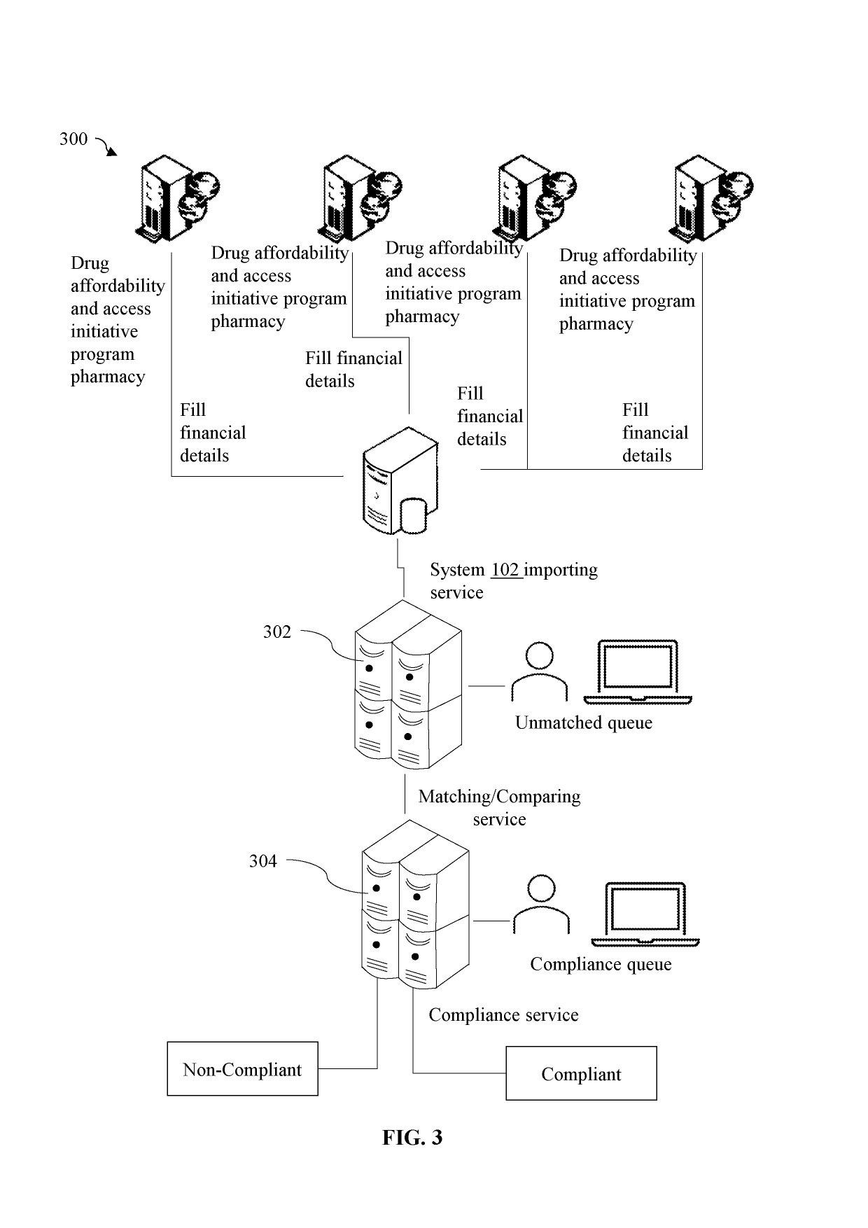

FIG. 3 illustrates an exemplary flow diagram 300 of the ML-based system 102 for validating the one or more prescription reconciliation parameters, in accordance with an embodiment of the present disclosure. The ML-based system 102 is configured to obtain the one or more data including at least one of: the information associated with the one or more patients whose prescriptions are filled, and the one or more metadata of the one or more filled prescriptions, from one or more pharmacies.

The ML-based system 102 is configured to match the one or more filled prescriptions obtained from the one or more pharmacies, with the one or more prescriptions stored in the one or more EMR systems 116. If the one or more filled prescriptions obtained from the one or more pharmacies, are not matched with the one or more prescriptions stored in the one or more EMR systems 116, then a comparison/matching service 302, as shown in FIG. 3, may be configured to adapt the manual review within the matching queue, where the workforce manually reviews for the correct match, across the ML-based system 102.

A compliance service 304, as shown in FIG. 3, is configured to determine whether each data of the one or more data associated with the one or more filled prescriptions are compliant with the drug affordability and access initiative program, based on the matching of the one or more filled prescriptions obtained from the one or more pharmacies, with the one or more prescriptions stored in the one or more EMR systems 116 to validate the one or more prescriptions.

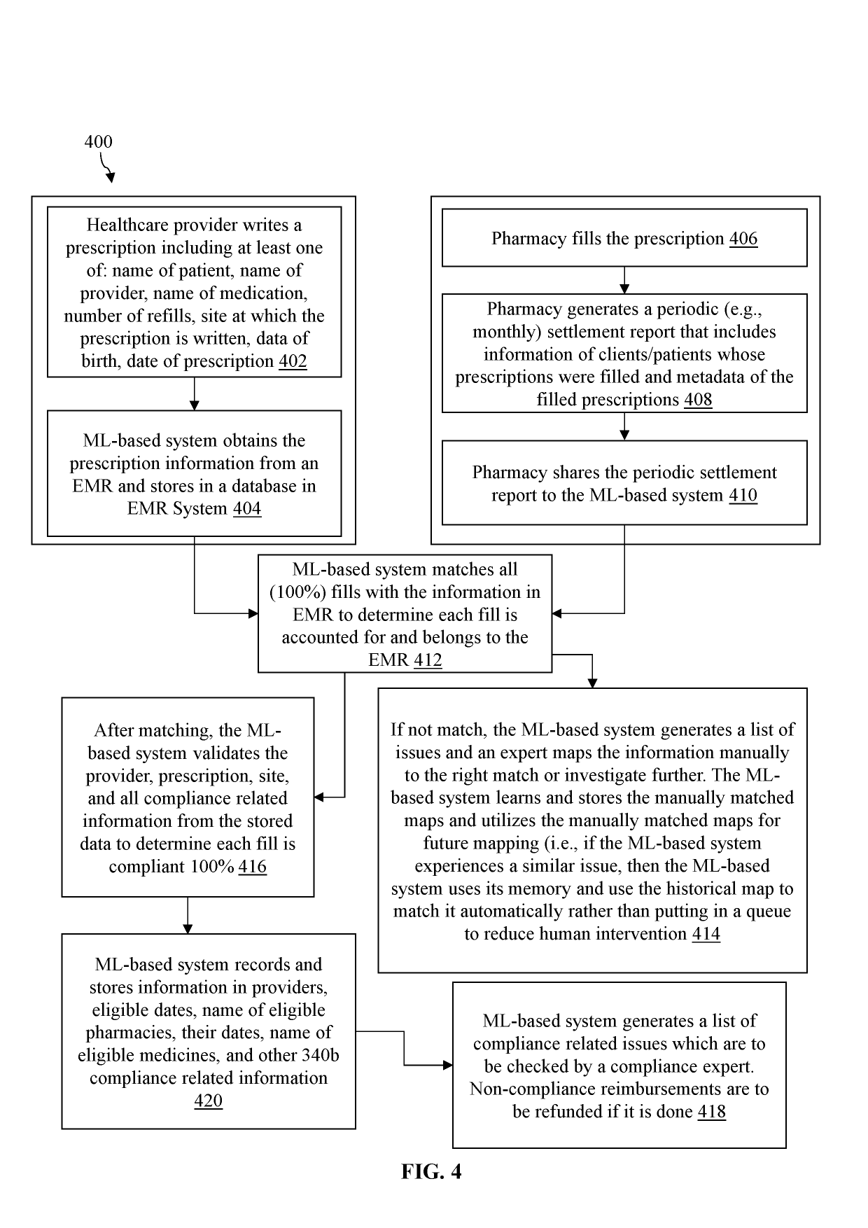

FIG. 4 illustrates a process flow 400 of the ML-based system 102 for validating the one or more prescriptions associated with the one or more patients, in accordance with an embodiment of the present disclosure. The one or more healthcare providers generate/write the one or more prescriptions and stores in the one or more databases 104, as shown in step 402. At step 404, the ML-based system 102 is configured to obtain the one or more prescription including the one or more information, from one or more databases 104. In an embodiment, the one or more information in the one or more prescriptions include at least one of: name of the one or more patients, date of birth of the one or more patients, one or more dates on which the one or more prescriptions are generated, name of the one or more healthcare providers, name of one or more medication, quantity of the one or more medication, number of refills, and one or more sites at which one or more prescriptions are generated.

At step 406, the one or more pharmacies are configured to fill the one or more prescriptions. At step 408, the one or more pharmacies are configured to generate the one or more periodic settlement reports (e.g., the monthly settlement report) with the one or more data including at least one of: the information associated with the one or more patients whose prescriptions are filled, and the one or more metadata of the one or more filled prescriptions. At step 410, the one or more pharmacies are configured to share the one or more periodic settlement reports to the ML-based system 102.

At step 412, the ML-based system 102 is configured to match the one or more filled prescriptions (100% filled prescriptions) obtained from the one or more pharmacies, with the one or more prescriptions stored in the one or more EMR systems 116. At step 414, the ML-based system 102 is configured to generate the list of issues associated with mappings of the one or more filled prescriptions obtained from the one or more pharmacies, with the one or more prescriptions stored in the one or more EMR systems 116, for the one or more users and adapt the one or more users to map the one or more filled prescriptions obtained from the one or more pharmacies, with the one or more prescriptions stored in the one or more EMR systems 116, manually, when the one or more filled prescriptions obtained from the one or more pharmacies, are not matched with the one or more prescriptions stored in the one or more EMR systems 116.

Further, the ML-based system 102 is configured to learn manually matched mappings of the one or more filled prescriptions obtained from the one or more pharmacies, with the one or more prescriptions stored in the one or more EMR systems 116. The ML-based system 102 is configured to store information associated with the manually matched mappings of the one or more filled prescriptions obtained from the one or more pharmacies, with the one or more prescriptions stored in the one or more EMR systems 116, in a memory (i.e., the one or more databases 104 of the ML-based system 102).

The ML-based system 102 is configured to utilize the stored information (i.e., historical information associated with the mappings) associated with the manually matched mappings of the one or more filled prescriptions obtained from the one or more pharmacies, with the one or more prescriptions stored in the one or more EMR systems 116, from the memory when the one or more filled prescriptions obtained from the one or more pharmacies, with the one or more prescriptions stored in the one or more EMR systems 116, are not matched. In other words, the ML-based system 102 is configured to utilize the historical information associated with the mappings to match the mappings automatically rather than putting in the queue to reduce the human work/intervention.

At step 416, the ML-based system 102 is configured to validate whether each data of the one or more data associated with the one or more filled prescriptions are compliant with the drug affordability and access initiative program, based on the matching of the one or more filled prescriptions obtained from the one or more pharmacies, with the one or more prescriptions stored in the one or more EMR systems 116. At step 418, the ML-based system 102 is configured to generate the list of compliance related issues which are to be determined by the one or more users (e.g., one or more compliance experts). The non-compliance reimbursements are refunded upon determination of the one or more prescriptions are non-compliant to the drug affordability and access initiative program.

At step 420, the ML-based system 102 is configured to store one or more information associated with at least one of: the one or more healthcare providers, qualified dates of the one or more healthcare providers, the one or more sites at which the one or more prescriptions are generated, one or more qualified pharmacies, dates of the one or more qualified pharmacies, name of one or more qualified medicines, and information associated with the one or more filled prescriptions being compliant with the drug affordability and access initiative program.

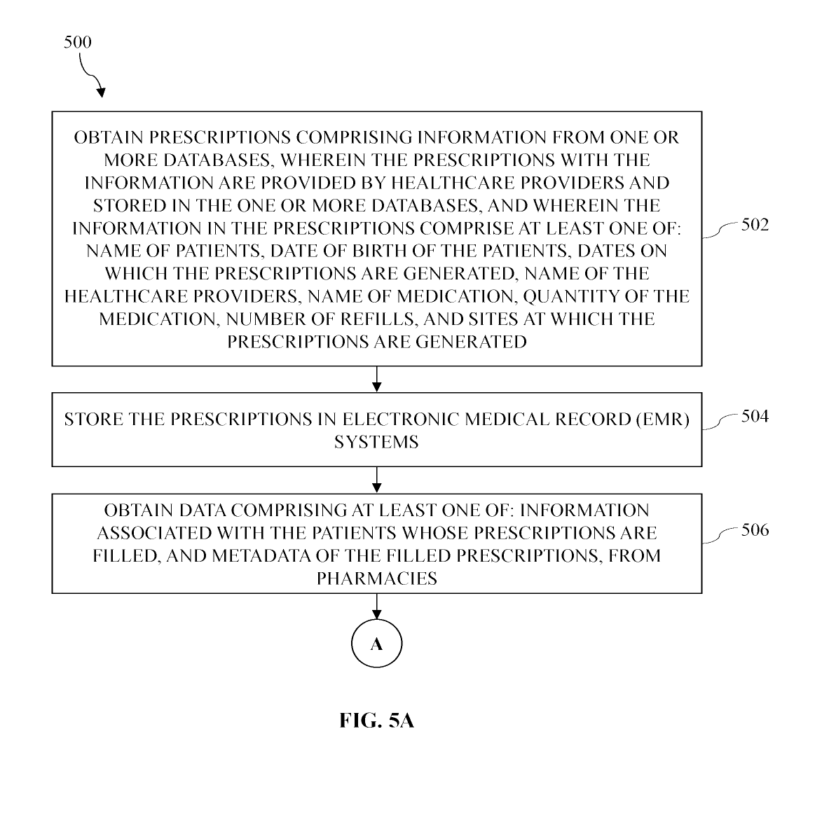

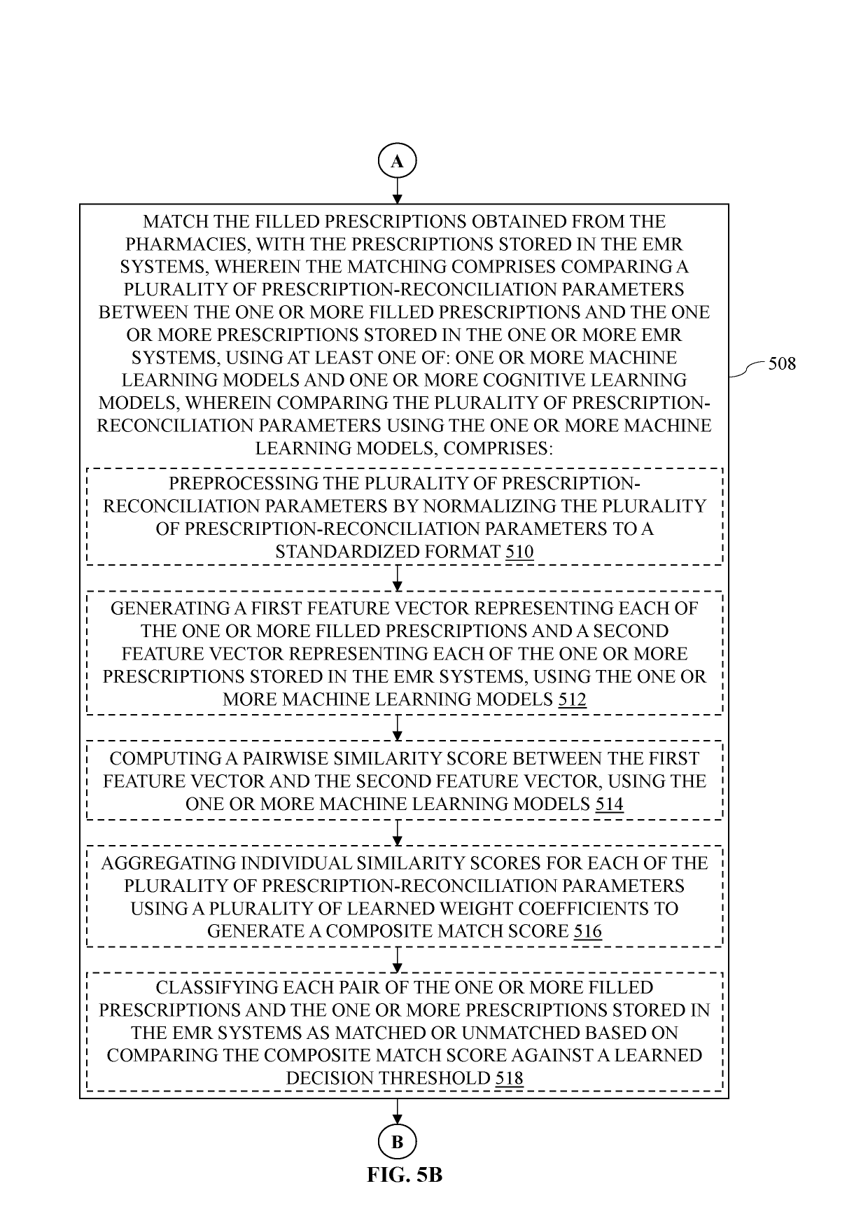

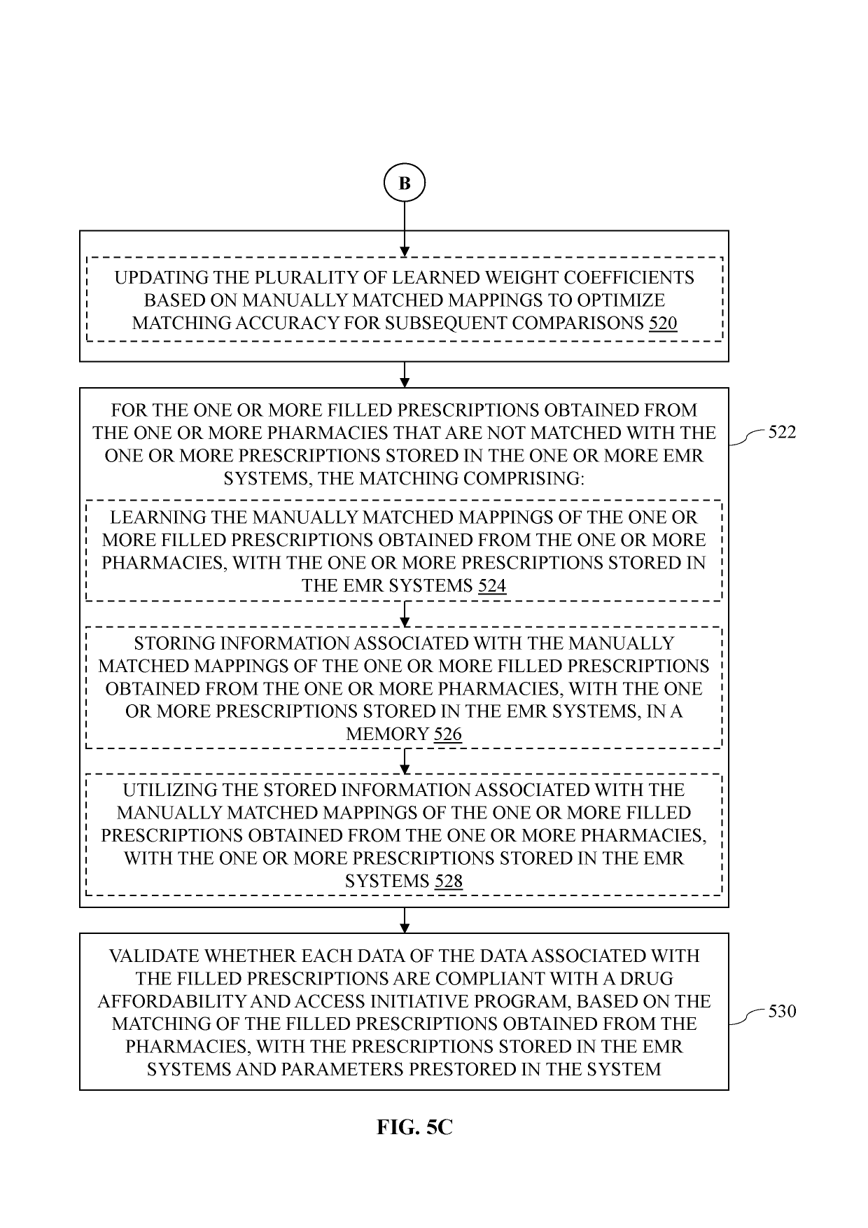

FIG. 5A-5C illustrates a flow chart illustrating a ML-based method 500 for validating the one or more prescriptions associated with the one or more patients, in accordance with an embodiment of the present disclosure.

At step 502, the one or more prescriptions include the one or more information, are obtained from the one or more databases 104. The one or more prescriptions with the one or more information are generated by one or more healthcare providers and stored in the one or more databases 104. In an embodiment, the one or more information in the one or more prescriptions may include at least one of: name of the one or more patients, date of birth of the one or more patients, one or more dates on which the one or more prescriptions are generated, name of the one or more healthcare providers, name of one or more medication, quantity of the one or more medication, number of refills, and one or more sites at which one or more prescriptions are generated.

At step 504, the one or more prescriptions are stored in the one or more electronic medical record (EMR) systems 116.

At step 506, the one or more data including at least one of: the information associated with the one or more patients whose prescriptions are filled, and one or more metadata of the one or more filled prescriptions, are obtained from the one or more pharmacies.

At step 508, the one or more filled prescriptions obtained from the one or more pharmacies, are matched with the one or more prescriptions stored in the one or more EMR systems 116. For matching the one or more filled prescriptions obtained from the one or more pharmacies, with the one or more prescriptions stored in the one or more EMR systems 116, the ML-based method 500 further comprises comparing the plurality of prescription-reconciliation parameters between the one or more filled prescriptions and the one or more prescriptions stored in the one or more EMR systems 116, using at least one of: the one or more machine learning models and the one or more cognitive learning models.

For comparing the plurality of prescription-reconciliation parameters between the one or more filled prescriptions and the one or more prescriptions stored in the one or more EMR systems 116, using the one or more machine learning models, the ML-based method 500 comprises: (a) preprocessing, as shown in step 510, the plurality of prescription-reconciliation parameters by normalizing the plurality of prescription-reconciliation parameters to a standardized format, wherein the plurality of prescription-reconciliation parameters comprise at least one of: a medication description normalized to one or more text fields, and dosage and quantity converted to uniform numerical units, (b) generating, as shown in step 512, a first feature vector representing each of the one or more filled prescriptions and a second feature vector representing each of the one or more prescriptions stored in the one or more EMR systems 116, using the one or more machine learning models, (c) computing, as shown in step 514, a pairwise similarity score between the first feature vector and the second feature vector, using the one or more machine learning models, (d) aggregating, as shown in step 516, individual similarity scores for each of the plurality of prescription-reconciliation parameters using a plurality of learned weight coefficients to generate a composite match score, (e) classifying, as shown in step 518, each pair of the one or more filled prescriptions and the one or more prescriptions stored in the one or more EMR systems 116 as matched or unmatched based on comparing the composite match score against a learned decision threshold, and (f) updating, as shown in step 520, the plurality of learned weight coefficients based on manually matched mappings to optimize matching accuracy for subsequent comparisons. At step 522, for the one or more filled prescriptions obtained from the one or more pharmacies that are not matched with the one or more prescriptions stored in the one or more EMR systems 116, the ML-based method 500 further comprises: (a) learning, as shown in step 524, the manually matched mappings of the one or more filled prescriptions obtained from the one or more pharmacies, with the one or more prescriptions stored in the one or more EMR systems 116, (b) storing, as shown in step 526, information associated with the manually matched mappings of the one or more filled prescriptions obtained from the one or more pharmacies, with the one or more prescriptions stored in the one or more EMR systems 116, in a memory, and (c) utilizing, as shown in step 528, the stored information associated with the manually matched mappings of the one or more filled prescriptions obtained from the one or more pharmacies, with the one or more prescriptions stored in the one or more EMR systems 116.

At step 530, the ML-based system 102 validates whether each data of the one or more data associated with the one or more filled prescriptions are compliant with the drug affordability and access initiative program, based on the matching of the one or more filled prescriptions obtained from the one or more pharmacies, with the one or more prescriptions stored in the one or more EMR systems 116 and one or more eligibility parameters prestored in the one or more EMR systems 116.

In FIG. 5A-5C, the circular symbols with “A and B” written inside are being used as off-page connectors. These are used for indicating that FIG. 5A continues to the subsequent pages as FIG. 5B-5C.

Numerous advantages of the present disclosure may be apparent from the discussion above. In accordance with the present disclosure, the ML-based system 102 for validating the one or more prescriptions is disclosed. By leveraging the capabilities of the ML-based system 102 for prescription reconciliation and compliance monitoring, the healthcare facilities/entities ensure accurate recording of the prescription fills and adherence to the drug affordability and access initiative program requirements.

The ML-based system 102 assists in maximizing financial benefits derived from the drug affordability and access initiative program and supports a financial sustainability of the participating healthcare facilities. The patients served by the healthcare facilities participating in the drug affordability and access initiative program benefit from reduced out-of-pocket costs for the medications. The discounted prices obtained through the drug affordability and access initiative program make the essential medications more affordable and accessible for the patients, particularly those with limited financial means or without adequate insurance coverage.

The ML-based system 102 is configured to effectively correlate the patient data, medication data, and pharmacy data between the pharmacy records and the one or more EMR systems 116. The ML-based system 102 is configured to streamline the prescription reconciliation process, minimize manual labor, reduce the errors, ensure compliance with regulatory requirements such as the drug affordability and access initiative program, and provide real-time insights for improved decision-making.

By automating the comparison process, the ML-based system 102 significantly reduces the number of human hours required to compare the one or more prescriptions between the one or more pharmacy records and the one or more EMR systems 116. By employing the one or more algorithms for the comparison process, the ML-based system 102 minimizes the risk of errors, ensuring that the one or more prescription reconciliation parameters is accurately recorded and reconciled.

By providing a foolproof method for reconciling the prescription fills and validating the compliance with the drug affordability and access initiative program guidelines, the ML-based system 102 assists in mitigating the risk of non-compliance and associated financial penalties. This proactive approach to the compliance management safeguards the financial integrity of the healthcare facilities and preserves its eligibility to participate in the drug affordability and access initiative program.

The written description describes the subject matter herein to enable any person skilled in the art to make and use the embodiments. The scope of the subject matter embodiments is defined by the claims and may include other modifications that occur to those skilled in the art. Such other modifications are intended to be within the scope of the claims if they have similar elements that do not differ from the literal language of the claims or if they include equivalent elements with insubstantial differences from the literal language of the claims.

The embodiments herein can comprise hardware and software elements. The embodiments that are implemented in software include but are not limited to, firmware, resident software, microcode, etc. The functions performed by various modules described herein may be implemented in other modules or combinations of other modules. For the purposes of this description, a computer-usable or computer-readable medium can be any apparatus that can comprise, store, communicate, propagate, or transport the program for use by or in connection with the instruction execution system, apparatus, or device.

The medium can be an electronic, magnetic, optical, electromagnetic, infrared, or semiconductor system (or apparatus or device) or a propagation medium. Examples of a computer-readable medium include a semiconductor or solid-state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W) and DVD.

Input/output (I/O) devices (including but not limited to keyboards, displays, pointing devices, etc.) can be coupled to the ML-based system 102 either directly or through intervening I/O controllers. Network adapters may also be coupled to the ML-based system 102 to enable the data processing system to become coupled to other data processing systems or remote printers or storage devices through intervening private or public networks. Modems, cable modem and Ethernet cards are just a few of the currently available types of network adapters.

A representative hardware environment for practicing the embodiments may include a hardware configuration of an information handling/ML-based system 102 in accordance with the embodiments herein. The ML-based system 102 herein comprises at least one processor or central processing unit (CPU). The CPUs are interconnected via the system bus 202 to various devices including at least one of: a random-access memory (RAM), read-only memory (ROM), and an input/output (I/O) adapter. The I/O adapter can connect to peripheral devices, including at least one of: disk units and tape drives, or other program storage devices that are readable by the ML-based system 102. The ML-based system 102 can read the inventive instructions on the program storage devices and follow these instructions to execute the methodology of the embodiments herein.

The ML-based system 102 further includes a user interface adapter that connects a keyboard, mouse, speaker, microphone, and/or other user interface devices including a touch screen device (not shown) to the bus to gather user input. Additionally, a communication adapter connects the bus to a data processing network, and a display adapter connects the bus to a display device which may be embodied as an output device including at least one of: a monitor, printer, or transmitter, for example.

A description of an embodiment with several components in communication with each other does not imply that all such components are required. On the contrary, a variety of optional components are described to illustrate the wide variety of possible embodiments of the invention. When a single device or article is described herein, it will be apparent that more than one device/article (whether or not they cooperate) may be used in place of a single device/article. Similarly, where more than one device or article is described herein (whether or not they cooperate), it will be apparent that a single device/article may be used in place of the more than one device or article, or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments. Also, the words “comprising,” “having,” “containing,” and “including,” and other similar forms are intended to be equivalent in meaning and be open-ended in that an item or items following any one of these words is not meant to be an exhaustive listing of such item or items or meant to be limited to only the listed item or items. It must also be noted that as used herein and in the appended claims, the singular forms “a,” “an,” and “the” include plural references unless the context clearly dictates otherwise.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that are issued on an application based here on. Accordingly, the embodiments of the present invention are intended to be illustrative, but not limiting, of the scope of the invention, which is set forth in the following claims.

What is claimed is:

1. A machine learning (ML)-based method for validating one or more prescriptions associated with one or more patients, the ML-based method comprising:
obtaining, by one or more hardware processors, the one or more prescriptions comprising one or more information, from one or more databases, wherein the one or more prescriptions with the one or more information are generated by one or more healthcare providers and stored in the one or more databases,
wherein the one or more information in the one or more prescriptions comprise at least one of: name of the one or more patients, date of birth of the one or more patients, one or more dates on which the one or more prescriptions are generated, name of the one or more healthcare providers, name of one or more medication, quantity of the one or more medication, number of refills, and one or more sites at which one or more prescriptions are generated;
storing, by the one or more hardware processors, the one or more prescriptions in one or more electronic medical record (EMR) systems;
obtaining, by the one or more hardware processors, one or more data comprising at least one of: information associated with the one or more patients whose prescriptions are filled, and one or more metadata of the one or more filled prescriptions, from one or more pharmacies;
matching, by the one or more hardware processors, the one or more filled prescriptions obtained from the one or more pharmacies, with the one or more prescriptions stored in the one or more EMR systems, wherein the matching comprises:
comparing, by the one or more hardware processors, a plurality of prescription-reconciliation parameters between the one or more filled prescriptions and the one or more prescriptions stored in the one or more EMR systems, using at least one of: one or more machine learning models and one or more cognitive learning models,
wherein comparing the plurality of prescription-reconciliation parameters between the one or more filled prescriptions and the one or more prescriptions stored in the one or more EMR systems, using the one or more machine learning models, comprises:
preprocessing, by the one or more hardware processors, the plurality of prescription-reconciliation parameters by normalizing the plurality of prescription-reconciliation parameters to a standardized format, wherein the plurality of prescription-reconciliation parameters comprise at least one of: a medication description normalized to one or more text fields, and dosage and quantity converted to uniform numerical units;
generating, by the one or more hardware processors, a first feature vector representing each of the one or more filled prescriptions and a second feature vector representing each of the one or more prescriptions stored in the one or more EMR systems, using the one or more machine learning models;
computing, by the one or more hardware processors, a pairwise similarity score between the first feature vector and the second feature vector, using the one or more machine learning models;
aggregating, by the one or more hardware processors, individual similarity scores for each of the plurality of prescription-reconciliation parameters using a plurality of learned weight coefficients to generate a composite match score;
classifying, by the one or more hardware processors, each pair of the one or more filled prescriptions and the one or more prescriptions stored in the one or more EMR systems as matched or unmatched based on comparing the composite match score against a learned decision threshold; and
updating, by the one or more hardware processors, the plurality of learned weight coefficients based on manually matched mappings to optimize matching accuracy for subsequent comparisons,
wherein at least one of: the one or more machine learning models and the one or more cognitive learning models, are configured to analyze the plurality of prescription-reconciliation parameters to identify similarities between the one or more filled prescriptions obtained from the one or more pharmacies and the one or more prescriptions stored in the one or more EMR systems, and
wherein, for the one or more filled prescriptions obtained from the one or more pharmacies that are not matched with the one or more prescriptions stored in the one or more EMR systems, the matching comprising:
learning, by the one or more hardware processors, the manually matched mappings of the one or more filled prescriptions obtained from the one or more pharmacies, with the one or more prescriptions stored in the one or more EMR systems;
storing, by the one or more hardware processors, information associated with the manually matched mappings of the one or more filled prescriptions obtained from the one or more pharmacies, with the one or more prescriptions stored in the one or more EMR systems, in a memory; and
utilizing, by the one or more hardware processors, the stored information associated with the manually matched mappings of the one or more filled prescriptions obtained from the one or more pharmacies, with the one or more prescriptions stored in the one or more EMR systems; and
validating, by the one or more hardware processors, whether each data of the one or more data associated with the one or more filled prescriptions are compliant with a drug affordability and access initiative program, based on the matching of the one or more filled prescriptions obtained from the one or more pharmacies, with the one or more prescriptions stored in the one or more EMR systems and one or more parameters prestored in the one or more EMR systems.

2. The ML-based method of claim 1, wherein comparing the plurality of prescription-reconciliation parameters between the one or more filled prescriptions and the one or more prescriptions stored in the one or more EMR systems, using the one or more cognitive learning models, comprises:
parsing, by the one or more hardware processors, the plurality of prescription-reconciliation parameters to extract one or more semantic entities comprising the medication description, the dosage, the quantity, and a unique identifier linked to each medication;
constructing, by the one or more hardware processors, a knowledge graph representation linking the extracted semantic entities from the one or more filled prescriptions with corresponding semantic entities from the one or more prescriptions stored in the one or more EMR systems, using the one or more cognitive learning models;
performing, by the one or more hardware processors, a semantic reasoning operation on the knowledge graph representation to infer equivalence relationships between the extracted semantic entities, wherein the semantic reasoning operation comprises identifying a generic medication name as equivalent to a corresponding brand-name medication based on a learned ontological relationship;
generating, by the one or more hardware processors, a semantic similarity score for each pair of the one or more filled prescriptions and the one or more prescriptions stored in the one or more EMR systems based on the inferred equivalence relationships; and
adapting, by the one or more hardware processors, the learned ontological relationships based on the manually matched mappings to optimize semantic reasoning accuracy for subsequent comparisons.

3. The ML-based method of claim 1, further comprising training, by the one or more hardware processors, the one or more machine learning models, by:
collecting, by the one or more hardware processors, a first training dataset comprising a plurality of labeled prescription pairs, wherein each labeled prescription pair comprises a filled prescription, a corresponding prescription from the one or more EMR systems, and a ground truth label indicating whether each prescription pair is a match or a non-match;
extracting, by the one or more hardware processors, a plurality of training feature vectors from the first training dataset, wherein each training feature vector encodes the plurality of prescription-reconciliation parameters;
initializing, by the one or more hardware processors, a plurality of weight coefficients corresponding to each of the plurality of prescription-reconciliation parameters;
computing, by the one or more hardware processors, a predicted similarity score for each labeled prescription pair by aggregating individual similarity scores using the plurality of weight coefficients;
computing, by the one or more hardware processors, a loss value representing a difference between the predicted similarity score and the ground truth label using a loss function;
computing, by the one or more hardware processors, a gradient of the loss value with respect to each of the plurality of weight coefficients using backpropagation; and
updating, by the one or more hardware processors, each of the plurality of weight coefficients by subtracting a product of a learning rate and the computed gradient from a current value of the weight coefficient.

4. The ML-based method of claim 2, further comprising training, by the one or more hardware processors, the one or more cognitive learning models, by:
ingesting, by the one or more hardware processors, a knowledge corpus comprising a plurality of medication ontologies and generic-to-brand name equivalence tables to establish foundational semantic relationships between medication entities;
constructing, by the one or more hardware processors, an initial ontology comprising a plurality of semantic nodes representing medication entities and a plurality of semantic edges representing relationships between the medication entities;
parsing, by the one or more hardware processors, a second training dataset comprising the plurality of labeled prescription pairs to extract semantic entities comprising the medication description, the dosage, the quantity, and the unique identifier linked to each medication;
populating, by the one or more hardware processors, a knowledge graph with the extracted semantic entities and inferred relationships;
learning, by the one or more hardware processors, a plurality of inference rules from the second training dataset, wherein each inference rule defines a logical condition under which two medication entities are determined to be equivalent;
validating, by the one or more hardware processors, the learned inference rules by performing semantic reasoning on the knowledge graph and comparing inferred equivalence relationships against ground truth labels;
refining, by the one or more hardware processors, the ontological relationships in the knowledge graph based on misclassified prescription pairs; and
adapting, by the one or more hardware processors, the ontological relationships based on the manually matched mappings by adding new semantic edges linking the medication entities that are manually determined to be equivalent.

5. The ML-based method of claim 1, further comprising:
adapting, by the one or more hardware processors, the one or more pharmacies to generate one or more periodic settlement reports with the one or more data comprising at least one of: the information associated with the one or more patients whose prescriptions are filled, and the one or more metadata of the one or more filled prescriptions.

6. The ML-based method of claim 1, further comprising:
storing, by the one or more hardware processors, one or more information associated with at least one of: the one or more healthcare providers, qualified dates of the one or more healthcare providers, the one or more sites at which the one or more prescriptions are generated, one or more qualified pharmacies, dates of the one or more qualified pharmacies, name of one or more qualified medicines, and information associated with the one or more filled prescriptions being compliant with the drug affordability and access initiative program.

7. The ML-based method of claim 1, further comprising triggering, by the one or more hardware processors, one or more events indicating that the one or more prescriptions are logged when the one or more information associated with the one or more prescriptions are compared.

8. The ML-based method of claim 1, further comprising:
generating, by the one or more hardware processors, one or more alerts for one or more issues associated with non-compliance of the one or more prescriptions with the drug affordability and access initiative program;
performing, by the one or more hardware processors, a compliance checking process when the one or more prescriptions are non-compliant with the drug affordability and access initiative program, wherein the compliance checking process comprises reviewing, by the one or more hardware processors, one or more discrepancies caused by one or more non-compliance prescriptions to determine an adherence to requirements and regulations associated with the drug affordability and access initiative program; and
performing, by the one or more hardware processors, one or more actions comprising at least one of: reversing of one or more transactions and implementation of one or more corrective measures to maintain the compliance of the one or more prescriptions with the drug affordability and access initiative program, upon identifying the one or more issues associated with the compliance of the one or more prescriptions with the drug affordability and access initiative program.

9. The ML-based method of claim 1, further comprising:
analyzing, by the one or more hardware processors, one or more financial data to validate the one or more financial data to be compliance with the drug affordability and access initiative program, wherein analyzing the one or more financial data comprises assessing one or more factors comprising at least one of: provider eligibility, program eligibility, and location eligibility; and
analyzing, by the one or more hardware processors, the one or more factors to determine whether at least one of: the one or more prescriptions and the one or more financial data are compliant with the drug affordability and access initiative program.

10. The ML-based method of claim 1, further comprising:
generating, by the one or more hardware processors, a list of issues associated with mappings of the one or more filled prescriptions obtained from the one or more pharmacies, with the one or more prescriptions stored in the one or more EMR systems, for one or more users;
adapting, by the one or more hardware processors, the one or more users to map the one or more filled prescriptions obtained from the one or more pharmacies, with the one or more prescriptions stored in the one or more EMR systems, when the one or more filled prescriptions obtained from the one or more pharmacies, are not matched with the one or more prescriptions stored in the one or more EMR systems.

11. A machine learning (ML)-based system for validating one or more prescriptions associated with one or more patients, the ML-based system comprising:
one or more hardware processors;
a memory coupled to the one or more hardware processors, wherein the memory comprises a plurality of subsystems in form of programmable instructions executable by the one or more hardware processors, and wherein the plurality of subsystems comprises:
a data obtaining subsystem configured to obtain the one or more prescriptions comprising one or more information, from one or more databases, wherein the one or more prescriptions with the one or more information are generated by one or more healthcare providers and stored in the one or more databases,
wherein the one or more information in the one or more prescriptions comprise at least one of: name of the one or more patients, date of birth of the one or more patients, one or more dates on which the one or more prescriptions are generated, name of the one or more healthcare providers, name of one or more medication, quantity of the one or more medication, number of refills, and one or more sites at which one or more prescriptions are generated;
a storage subsystem configured to store the one or more prescriptions in one or more electronic medical record (EMR) systems;
the data obtaining subsystem configured to obtain one or more data comprising at least one of: information associated with the one or more patients whose prescriptions are filled, and one or more metadata of the one or more filled prescriptions, from one or more pharmacies;
a prescription matching subsystem configured to match the one or more filled prescriptions obtained from the one or more pharmacies, with the one or more prescriptions stored in the one or more EMR systems,
wherein in matching the plurality of prescription-reconciliation parameters between the one or more filled prescriptions and the one or more prescriptions stored in the one or more EMR systems, using the one or more machine learning models, the prescription matching subsystem is further configured to compare a plurality of prescription-reconciliation parameters between the one or more filled prescriptions and the one or more prescriptions stored in the one or more EMR systems, using at least one of: one or more machine learning models and one or more cognitive learning models,
wherein in comparing the plurality of prescription-reconciliation parameters between the one or more filled prescriptions and the one or more prescriptions stored in the one or more EMR systems, using the one or more machine learning models, the prescription matching subsystem is further configured to:
preprocess the plurality of prescription-reconciliation parameters by normalizing the plurality of prescription-reconciliation parameters to a standardized format, wherein the plurality of prescription-reconciliation parameters comprise at least one of: a medication description normalized to one or more text fields, and dosage and quantity converted to uniform numerical units;
generate a first feature vector representing each of the one or more filled prescriptions and a second feature vector representing each of the one or more prescriptions stored in the one or more EMR systems, using the one or more machine learning models;
compute a pairwise similarity score between the first feature vector and the second feature vector, using the one or more machine learning models;
aggregate individual similarity scores for each of the plurality of prescription-reconciliation parameters using a plurality of learned weight coefficients to generate a composite match score;
classify each pair of the one or more filled prescriptions and the one or more prescriptions stored in the one or more EMR systems as matched or unmatched based on comparing the composite match score against a learned decision threshold; and
update the plurality of learned weight coefficients based on manually matched mappings to optimize matching accuracy for subsequent comparisons,
wherein at least one of: the one or more machine learning models and the one or more cognitive learning models, are configured to analyze the plurality of prescription-reconciliation parameters to identify similarities between the one or more filled prescriptions obtained from the one or more pharmacies and the one or more prescriptions stored in the one or more EMR systems, and
wherein, for the one or more filled prescriptions obtained from the one or more pharmacies that are not matched with the one or more prescriptions stored in the one or more EMR systems, the prescription matching subsystem is further configured to:
learn the manually matched mappings of the one or more filled prescriptions obtained from the one or more pharmacies, with the one or more prescriptions stored in the one or more EMR systems;
store information associated with the manually matched mappings of the one or more filled prescriptions obtained from the one or more pharmacies, with the one or more prescriptions stored in the one or more EMR systems, in a memory; and
utilize the stored information associated with the manually matched mappings of the one or more filled prescriptions obtained from the one or more pharmacies, with the one or more prescriptions stored in the one or more EMR systems; and
a prescription validating subsystem configured to validate whether each data of the one or more data associated with the one or more filled prescriptions are compliant with a drug affordability and access initiative program, based on the matching of the one or more filled prescriptions obtained from the one or more pharmacies, with the one or more prescriptions stored in the one or more EMR systems and one or more parameters prestored in the one or more EMR systems.

12. The ML-based system of claim 11, wherein in comparing the plurality of prescription-reconciliation parameters between the one or more filled prescriptions and the one or more prescriptions stored in the one or more EMR systems, using the one or more cognitive learning models, the prescription matching subsystem is further configured to:
parse the plurality of prescription-reconciliation parameters to extract one or more semantic entities comprising the medication description, the dosage, the quantity, and a unique identifier linked to each medication;
construct a knowledge graph representation linking the extracted semantic entities from the one or more filled prescriptions with corresponding semantic entities from the one or more prescriptions stored in the one or more EMR systems, using the one or more cognitive learning models;
perform a semantic reasoning operation on the knowledge graph representation to infer equivalence relationships between the extracted semantic entities, wherein the semantic reasoning operation comprises identifying a generic medication name as equivalent to a corresponding brand-name medication based on a learned ontological relationship;
generate a semantic similarity score for each pair of the one or more filled prescriptions and the one or more prescriptions stored in the one or more EMR systems based on the inferred equivalence relationships; and
adapt the learned ontological relationships based on the manually matched mappings to optimize semantic reasoning accuracy for subsequent comparisons.

13. The ML-based system of claim 11, further comprising a training subsystem configured t train the one or more machine learning models, by:
collecting a first training dataset comprising a plurality of labeled prescription pairs, wherein each labeled prescription pair comprises a filled prescription, a corresponding prescription from the one or more EMR systems, and a ground truth label indicating whether each prescription pair is a match or a non-match;
extracting a plurality of training feature vectors from the first training dataset, wherein each training feature vector encodes the plurality of prescription-reconciliation parameters;
initializing a plurality of weight coefficients corresponding to each of the plurality of prescription-reconciliation parameters;
computing a predicted similarity score for each labeled prescription pair by aggregating individual similarity scores using the plurality of weight coefficients;
computing a loss value representing a difference between the predicted similarity score and the ground truth label using a loss function;
computing a gradient of the loss value with respect to each of the plurality of weight coefficients using backpropagation; and
updating each of the plurality of weight coefficients by subtracting a product of a learning rate and the computed gradient from a current value of the weight coefficient.

14. The ML-based system of claim 12, wherein the training subsystem is further configured to train the one or more cognitive learning models, by:
ingesting a knowledge corpus comprising a plurality of medication ontologies and generic-to-brand name equivalence tables to establish foundational semantic relationships between medication entities;
constructing an initial ontology comprising a plurality of semantic nodes representing medication entities and a plurality of semantic edges representing relationships between the medication entities;
parsing a second training dataset comprising the plurality of labeled prescription pairs to extract semantic entities comprising the medication description, the dosage, the quantity, and the unique identifier linked to each medication;
populating a knowledge graph with the extracted semantic entities and inferred relationships;
learning a plurality of inference rules from the second training dataset, wherein each inference rule defines a logical condition under which two medication entities are determined to be equivalent;
validating the learned inference rules by performing semantic reasoning on the knowledge graph and comparing inferred equivalence relationships against ground truth labels;
refining the ontological relationships in the knowledge graph based on misclassified prescription pairs; and
adapting the ontological relationships based on the manually matched mappings by adding new semantic edges linking the medication entities that are manually determined to be equivalent.

15. The ML-based system of claim 11, wherein the one or more pharmacies are configured to generate one or more periodic settlement reports with the one or more data comprising at least one of: the information associated with the one or more patients whose prescriptions are filled, and the one or more metadata of the one or more filled prescriptions.

16. The ML-based system of claim 11, wherein the storage subsystem is further configured to store one or more information associated with at least one of: the one or more healthcare providers, qualified dates of the one or more healthcare providers, the one or more sites at which the one or more prescriptions are generated, one or more qualified pharmacies, dates of the one or more qualified pharmacies, name of one or more qualified medicines, and information associated with the one or more filled prescriptions being compliant with the drug affordability and access initiative program.

17. The ML-based system of claim 11, further comprising an alerts generating subsystem configured to:
generate one or more alerts for one or more issues associated with non-compliance of the one or more prescriptions with the drug affordability and access initiative program;
perform a compliance checking process when the one or more prescriptions are non-compliant with the drug affordability and access initiative program, wherein the compliance checking process comprises reviewing one or more discrepancies caused by one or more non-compliance prescriptions to determine an adherence to requirements and regulations associated with the drug affordability and access initiative program; and
perform one or more actions comprising at least one of: reversing of one or more transactions and implementation of one or more corrective measures to maintain the compliance of the one or more prescriptions with the drug affordability and access initiative program, upon identifying the one or more issues associated with the compliance of the one or more prescriptions with the drug affordability and access initiative program.

18. The ML-based system of claim 11, wherein the prescription validating subsystem is further configured to:
analyze one or more financial data to validate the one or more financial data to be compliance with the drug affordability and access initiative program, wherein analyzing the one or more financial data comprises assessing one or more factors comprising at least one of: provider eligibility, program eligibility, and location eligibility; and
analyze the one or more factors to determine whether at least one of: the one or more prescriptions and the one or more financial data are compliant with the drug affordability and access initiative program.

19. The ML-based system of claim 11, wherein the prescription matching subsystem is further configured to:
generate a list of issues associated with mappings of the one or more filled prescriptions obtained from the one or more pharmacies, with the one or more prescriptions stored in the one or more EMR systems, for one or more users;
adapt the one or more users to map the one or more filled prescriptions obtained from the one or more pharmacies, with the one or more prescriptions stored in the one or more EMR systems, when the one or more filled prescriptions obtained from the one or more pharmacies, are not matched with the one or more prescriptions stored in the one or more EMR systems.

20. A non-transitory computer-readable storage medium having instructions stored therein that when executed by one or more hardware processors, cause the one or more hardware processors to execute operations of:
obtaining one or more prescriptions comprising one or more information, from one or more databases, wherein the one or more prescriptions with the one or more information are generated by one or more healthcare providers and stored in the one or more databases,
wherein the one or more information in the one or more prescriptions comprise at least one of: name of the one or more patients, date of birth of the one or more patients, one or more dates on which the one or more prescriptions are generated, name of the one or more healthcare providers, name of one or more medication, quantity of the one or more medication, number of refills, and one or more sites at which one or more prescriptions are generated;
storing the one or more prescriptions in one or more electronic medical record (EMR) systems;
obtaining one or more data comprising at least one of: information associated with the one or more patients whose prescriptions are filled, and one or more metadata of the one or more filled prescriptions, from one or more pharmacies;
matching the one or more filled prescriptions obtained from the one or more pharmacies, with the one or more prescriptions stored in the one or more EMR systems, wherein the matching comprises:
comparing a plurality of prescription-reconciliation parameters between the one or more filled prescriptions and the one or more prescriptions stored in the one or more EMR systems, using at least one of: one or more machine learning models and one or more cognitive learning models,
wherein comparing the plurality of prescription-reconciliation parameters between the one or more filled prescriptions and the one or more prescriptions stored in the one or more EMR systems, using the one or more machine learning models, comprises:
preprocessing the plurality of prescription-reconciliation parameters by normalizing the plurality of prescription-reconciliation parameters to a standardized format, wherein the plurality of prescription-reconciliation parameters comprise at least one of: a medication description normalized to one or more text fields, and dosage and quantity converted to uniform numerical units;
generating a first feature vector representing each of the one or more filled prescriptions and a second feature vector representing each of the one or more prescriptions stored in the one or more EMR systems, using the one or more machine learning models;
computing a pairwise similarity score between the first feature vector and the second feature vector, using the one or more machine learning models;
aggregating individual similarity scores for each of the plurality of prescription-reconciliation parameters using a plurality of learned weight coefficients to generate a composite match score;
classifying each pair of the one or more filled prescriptions and the one or more prescriptions stored in the one or more EMR systems as matched or unmatched based on comparing the composite match score against a learned decision threshold; and
updating the plurality of learned weight coefficients based on manually matched mappings to optimize matching accuracy for subsequent comparisons,
wherein at least one of: the one or more machine learning models and the one or more cognitive learning models, are configured to analyze the plurality of prescription-reconciliation parameters to identify similarities between the one or more filled prescriptions obtained from the one or more pharmacies and the one or more prescriptions stored in the one or more EMR systems, and
wherein, for the one or more filled prescriptions obtained from the one or more pharmacies that are not matched with the one or more prescriptions stored in the one or more EMR systems, the matching comprising:
learning the manually matched mappings of the one or more filled prescriptions obtained from the one or more pharmacies, with the one or more prescriptions stored in the one or more EMR systems;
storing information associated with the manually matched mappings of the one or more filled prescriptions obtained from the one or more pharmacies, with the one or more prescriptions stored in the one or more EMR systems, in a memory; and
utilizing the stored information associated with the manually matched mappings of the one or more filled prescriptions obtained from the one or more pharmacies, with the one or more prescriptions stored in the one or more EMR systems; and
validating whether each data of the one or more data associated with the one or more filled prescriptions are compliant with a drug affordability and access initiative program, based on the matching of the one or more filled prescriptions obtained from the one or more pharmacies, with the one or more prescriptions stored in the one or more EMR systems and one or more parameters prestored in the one or more EMR systems.
